(19) United States
(12) Patent Application Publication
(10) Pub. No.: US 2022/0401207 A1
(43) Pub. Date: Dec. 22, 2022

(54) DEVICES, SYSTEMS, AND METHODS FOR TREATMENT OF DUCT OCCLUSION

(71) Applicant: Purdue Research Foundation, West Lafayette, IN (US)

(72) Inventors: Pablo Daniel Zavattieri, West Lafayette, IN (US); Kristiaan William Hector, Shelby Township, MI (US)

(21) Appl. No.: 17/843,997

(22) Filed: Jun. 18, 2022

Related U.S. Application Data
(60) Provisional application No. 63/212,543, filed on Jun. 18, 2021.

(51) Int. Cl. A61F 2/04

(52) U.S. Cl. CPC A61F 2/04; A61F 2002/041; A61F 2210/0014; A61F 2210/008; A61F 2250/0067

(57) ABSTRACT

Stents comprising a first region and a second region are provided, where at least the second region comprises one or more phase transforming cellular materials configured to move the outlet between an open configuration and a closed configuration in response to certain triggers. Such stents can also comprise one or more analog for a shape memory alloy (ASMA) unit cells on an inner surface of the first region such that, in response to resistive forces, the ASMA unit cells exert controllable motion to clear the stent. Methods of treatment of cancer, jaundice, and other diseases are also provided.

### PRIORITY

The present application is related to and claims the priority benefit of U.S. Provisional Patent Application No. 63/212,543 filed Jun. 18, 2021, the content of which is hereby expressly incorporated by reference in its entirety into this disclosure.

### TECHNICAL FIELD

This disclosure relates to medical methods and devices for treating whole or partial biliary tract obstructions including those caused by, for example, a tumor or cancerous mass. In some embodiments, the present disclosure relates to a two-part biliary stent that can be inserted into the biliary tract and, a portion of which, seats within the ampulla of Vater to expand and shrink at the same rate as the Sphincter of Oddi to allow for the opening and closing thereof while the stent is deployed.

### BACKGROUND

The biliary tree is a system of organs and ducts that creates, stores, and transports bile. A biological one-way valve, known as the Sphincter of Oddi (SO), exists at the intersection of the common bile duct (CBD) (trunk of the biliary tree) and the duodenum which can control bile flow and block duodenal contents (e.g., bacteria). This valve is controlled via neurological, hormonal, and mechanical stimuli. Obstructions can hinder sphincter functionality and/or block the CBD, which can lead to serious complications including severe infection.

The presence of cancer—especially aggressive ones—can not only cause biliary tree obstructions (both partial and full), but can also present major complications in treatment. The two most common malignant neoplasms known to occlude the bile ducts are pancreatic ductal adenocarcinoma and primary bile duct cancer (cholangiocarcinoma or CC), however there are many others. Both pancreatic cancer and CC are notorious for presenting at an advanced stage where immediate surgery is contraindicated.

When a patient is diagnosed with CC or any other type of bile duct cancer, the biliary tree is first explored for respectable regions. If this fails, which is common due to these types of cancers (e.g., pancreatic and CC) characteristically presenting at an advanced stage, conventionally, a stent can be placed into the bile ducts to maintain bile flow and alleviate pain. Additionally, chemoradiotherapy is applied in an attempt to downstage the tumor.

Biliary stenting is one of the surgical treatment methods for treating both blocked and partially blocked biliary ducts. Generally, a tubular stent is installed to hold the ducts open when constricted or blocked by a cancerous mass (or otherwise) to thus facilitate the flow of bile through the lumen of the duct. FIGS. 1A and 1B illustrate how conventional stents can be used to, for example, hold open a pathway for bile flow.

There are several types of bile duct stents ranging from plastic stents to metallic stents encased in a polymeric sheathing, and the effectiveness of these designs vis-à-vis preventing bile leakage or obstruction in the bile duct varies. FIGS. 1C and 1D illustrate various examples of conventional stents including metallic mesh stents that are capable of self-expansion, plastic stents, drug eluting stents, and biodegradable stents (not shown). Plastic stents (e.g., FIG. 1D) are low cost and prevent bile leakage; however, they are susceptible to stent obstructions, which can lead to Jaundice. The more costly metallic stents (e.g., FIG. 1C) offer enhanced protection against bile leakage and allow for long patency (i.e. they can be left in the patient for longer periods of time without the need for removal/replacement).

However, stent insertion into the CBD results in the permanent opening of the SO (see FIG. 1A). As noted above, one of the primary purposes of the SO is to block the plethora of bacteria present within the duodenum from entering the CBD and causing infection. By way of example, Table 1 sets forth a list of pathological bacteria cultured from a sample taken from human patients experiencing Jaundice.

TABLE 1
Distribution of bacteria isolated from bile

| | Bile samples cultured (n = 36)* | | |
|---|---|---|---|
| | | | Co- |
| | Controls | N-acetyl-cysteine | trimoxazole |
| Bacteria | (n = 14) | (n = 12) | (n = 10) |
| Enterococcus species | 9 | 10 | 6 |
| Escherichia coli | 7 | 6 | —** |
| Klebsiella species | 7 | 8 | 5 |
| K. pneumonia | 4 | 3 | 4 |
| K. oxytoca | 3 | 5 | 1 |
| Enterobacter species | 4 | 6 | 2 |
| Streptococcus species | 5 | 4 | —** |
| Pseudomonas aeruginosa | 1 | — | 1 |
| Citrobacter freundii | 1 | 1 | — |
| Proteus species | — | 1 | — |
| Staphylococcus | — | — | 1 |
| epidermidis | | | |
| Clostridium species | 1 | 5 | 2 |
| Bacteroides fragilis | — | — | 1 |
| Candida species | 1 | — | 2 |

*All mixed cultures; includes four premature stent exchanges with clinical cholangitis (one control, two N-acetylcysteine, and one co-trimoxazole group),
**p < 0.05 (versus control and/or N-acetylcysteine group).

With the SO open, bacteria can make their way up the stent and, thus, the biliary tree, pancreas, and liver can become susceptible to infection. Despite the variety of biliary duct stents available, there is not a conventional stent that replicates the normal motor functionality of the SO and allows the biliary system to return to its natural functioning state; instead, conventional stent technology merely facilitates flow.

Stent devices are needed that enable flow through an obstructed biliary tract while concurrently replacing a dysfunctional SO and/or allowing for normal SO function as a means to combat not only bile duct cancer, but any type of bile duct compression or obstruction that can be treated with stent placement.

### SUMMARY

In certain embodiments, stents are provided. A stent can comprise a first region comprising an upstream end, a downstream end, and a lumen extending a length between the upstream end and the downstream end. The first region of the stent can have an elongated tubular configuration where each of the downstream end and the upstream end are expanded radially. Additionally, the first region can define a first diameter along the length of the lumen. The stent can further comprise a second region coupled with the downstream end of the first region and defining an outlet that is in fluid communication with the lumen of the first region. The second region can be comprised of one or more phase transforming cellular materials (PXCM) configured to move the outlet between an open configuration and a closed configuration in response to a change in one or more of an energy imbalance in the PXCM, a change in pressure through an interior of the second region, and a change in a local concentration of cholecystokinin (CCK). In certain embodiments of the stent, the stent moving between an open configuration and a closed configuration emulates the mechanics and associated geometric changes of an ampulla of Vater during contraction and relaxation of a Sphincter of Oddi (SO).

In certain embodiments, the first region further comprises a reduced configuration where each of the downstream end and the upstream end are collapsed relative to each other in the tubular configuration and the first region defines a second diameter along the length of the lumen. There, the second diameter can be less than the first diameter of the elongated tubular configuration.

The first region of the stent can be configured for self-expansion from the reduced configuration to the tubular configuration. In certain embodiments, the first region is configured to increase a stiffness when subjected to a circumferential load, a concentric radial force, or an eccentric radial force. In certain embodiments, the first region comprises one or more PXCM or architected material analog for shape memory alloy (ASMA) unit cells. The first region can further comprise a drug eluting stent.

The first region can further comprise a one-way valve. In certain embodiments where the first region comprises a one-way valve, the one-way valve comprises an interior surface defining the lumen and extending between the upstream end and the downstream end. There, the interior surface can comprise one or more interior walls of a fixed-geometry passive check valve configuration to permit free passage of fluid through the lumen in a first direction but deter or prevent back flow of the fluid in a direction opposite the first direction. In certain embodiments where the first region comprises a one-way valve, the first region further comprises at least one PXCM covering positioned around a circumference of the first region, each of the PXCM coverings configured to compress or decompress the underlying first region in response to a change in local concentration of CCK to restrict or allow, respectively, fluid flow through the first region.

The first region and/or the second region of the stent can be biodegradable.

In certain embodiments, an interior surface that defines the lumen of the first region can comprise one or more ASMA unit cells or two or more ASMA unit cells. Each ASMA unit cell has a wavelength of 35 mm, 40 mm, 50 mm, or 60 mm. In certain embodiments, the stent comprises a first region, but not a second region, and the lumen of the first region comprises an interior surface comprising one or more ASMA unit cells, or two or more ASMA unit cells. In certain embodiments, the interior surface of the first region comprises one or more sets of ASMA unit cells. The ASMA unit cells can respond to restrictive or compressive force and an increase in temperature with a reversal of displacement (i.e. pushing back against a force that deforms such ASMA unit cells).

In certain embodiments, a stent comprises a first region, a second region coupled with a downstream end of the first region, and at least one PXCM covering positioned around a circumference of the first region. The first region can comprise an upstream end, a downstream end, a lumen extending a length between the upstream end and the downstream end, and an interior surface extending between the upstream end and the downstream end and defining at least a portion of the lumen. The interior surface can comprise one or more interior walls of a fixed geometry passive check valve configured to permit free passage of fluid through the lumen in a downstream direction but deter or prevent back flow of the fluid in an upstream direction, and the first region can be movable between a tubular configuration having a first diameter and a reduced configuration having a second diameter. There, when the tubular configuration of each of the downstream end and the upstream end are expanded radially, in the reduced configuration each of the downstream end and the upstream end can be collapsed relative to each other in the tubular configuration and the second diameter is less than the first diameter.

The second region of the stent can define an outlet in fluid communication with the lumen of the first region, wherein the second region is comprised of one or more PXCM configured to move the outlet between an open configuration and a closed configuration in response to a change in one or more of an energy imbalance in the PXCM, a change in pressure through an interior of the second region, and a change in a local concentration of CCK.

The at least one PXCM covering positioned around a circumference of the first region can be configured to compress or decompress the underlying first region in response to a change in concentration of CCK to restrict or allow, respectively, fluid flow through the first region.

Methods for treating a subject having a wholly or partially compressed or obstructed duct are also provided. In certain embodiments, the method comprises providing any of the stents described herein (e.g., a self-expanding stent); inserting, or having inserted, the stent in a reduced configuration into a targeted duct of the subject; and expanding, or allowing to expand, the stent in the targeted duct. For example, the stent can comprise a first region comprising an upstream end, a downstream end, and a lumen extending a length between the upstream end and the downstream end, wherein the first region is movable between a tubular configuration having a first diameter and a reduced configuration having a second diameter, where in the tubular configuration each of the downstream end and the upstream end are expanded radially, in the reduced configuration each of the downstream end and the upstream end are collapsed relative to each other in the tubular configuration, and the second diameter is less than the first diameter, and a second region coupled with the downstream end of the first region, defining an outlet in fluid communication with the lumen of the first region, wherein the second region is comprised of one or more PXCM configured to move the outlet between an open configuration and a closed configuration in response to a change in one or more of an energy imbalance in the PXCM, a change in pressure through an interior of the second region, and a change in a local concentration of CCK.

In certain embodiments, the outlet of the second region of the stent moving between an open configuration and a closed configuration emulates the mechanics and associated geometric changes of a SO of the subject during contraction and relaxation.

In certain embodiments of the method, the targeted duct is a common bile duct and the method can further comprise positioning the second region of the stent (e.g., self-expanding stent) within an ampulla of Vater of the subject.

The step of inserting can be performed endoscopically. In certain embodiments, the targeted duct is wholly or partially compressed or obstructed by a cancerous mass or tumor. In certain embodiments, the method further comprises administering to the subject a treatment for the cancerous mass or tumor (e.g., chemotherapy or chemoradiotherapy).

### BRIEF DESCRIPTION OF DRAWINGS

The disclosed embodiments and other features, advantages, and aspects contained herein, and the matter of attaining them, will become apparent in light of the following detailed description of various exemplary embodiments of the present disclosure. Such detailed description will be better understood when taken in conjunction with the accompanying drawings, wherein:

FIGS. 1A and 1B show photographs of a stent placed in the common bile duct (CBD) to hold the Sphincter of Oddi (SO) open and to hold the pathway open through the lumen for bile flow;

FIGS. 1C and 1D illustrate examples of various conventional stent types including metallic mesh stents capable of self-expansion (FIG. 1C, left), plastic stents (FIG. 1D), drug eluting stents (FIG. 1D), and biodegradable stents (not shown).

FIG. 2A shows a schematic of hepatocyte (bile canaliculi) cells that line the intrahepatic bile ducts, which are tasked with the creation and secretion of bile.

FIG. 2B shows a schematic of the gastrointestinal system of a human being, which comprises the biliary tree that includes a series of organs and ducts that necessitate the creation, storage, transportation and release of bile into the duodenum.

FIG. 2C is a diagram displaying the bile salt-cholesterol-phospholipid (lecithin) phases and describing the different cholesterol solubilization and bile lithiation phases possible in bile.

FIG. 3 shows graphical data related to shear-rate dependency of four patients diagnosed with jaundice brought on by cholelithiasis (gall stones), which are not representative of normal physiological function.

FIG. 4A shows a schematic of the connections between the gall bladder, the cystic duct, and the common-hepatic and common bile ducts.

FIGS. 4B-4E show photographs of different perspectives of the lumen geometry of the cystic duct that comprises the spiral-shaped valves of Heister.

FIGS. 5A and 5B show two-dimensional (FIG. 5A) and three-dimensional (FIG. 5B) models of the complex lumen geometry of the cystic duct to observe how the flow resistance is affected by the lumen geometry and the Reynolds number.

FIG. 5C shows graphical data representing how bile specimens taken from two different subjects flowed through the 3D model of FIG. 5A with different numbers of baffles n and different Reynolds number.

FIG. 5D shows graphical data illustrating how normalized flow resistance increases with smaller baffle clearances (i.e. c/D) (see FIG. 5A) and larger Reynolds numbers.

FIG. 6 shows graphical data of a series of intraductal pressure readings taken in a study designed to assess the pressure gradient between the gall bladder and SO.

FIG. 7 shows graphical data supporting that compliance of the gall bladder varies significantly from subject to subject, with each curve representing a different subject.

FIG. 8A is a schematic illustration of a mammalian biliary system showing how bile drains down through the CBD, where, due to the pressure differential and the stiffness of the SO, bile is directed through the cystic duct and into the gall bladder for storage.

FIG. 8B shows a close-up schematic of the muscle regions of the SO, whose compliance and geometry allows or restricts bile flow and the opening and closing of the SO.

FIG. 8C illustrates the shape of the ampulla of Vater when the SO is open (i.e. the SO is compliant and allows for the flow of bile into the duodenum).

FIG. 8D shows the shape of the ampulla of Vater when the SO is closed (i.e. the SO is rigid and does not allow the flow of bile into the duodenum).

FIG. 9A shows a graphical representation of the motor function of the SO in different regions thereof, with motor function being characterized by rhythmic spikes in lumen pressure which range between 50-150 mm Hg above that of the duodenum and occur 2-5 times per minute; measurements of lumen pressure taken in three separate regions via catheter equipped with a pressure transducer: Cephalad (i.e. front, pertaining to the major papilla), Middle (i.e. pertaining to the ampulla), and Caudad (i.e. pertaining to the distal CBD, the region leading from the CBD to the ampulla).

FIG. 9B is a table of data that highlights the values of lumen pressure in the SO acceptable for healthy subjects in the middle two columns (labeled “Median” and “Range”) and values of lumen pressure that are not acceptable for healthy subjects in the column on the far right (labeled “Abnormal”).

FIG. 10A shows a schematic of an experimental apparatus used in a study for observing SO motor function in possum specimens, where each specimen was placed in a bath of Krebs solution and the pressure in the CBD of each specimen was gradually changed, allowing the Krebs solution to flow from the Inflow reservoir through the inflow catheter, heating coil and bubble trap, and finally through the specimen (duodenum pressure was regulated via the outflow pump at 0, 4, or 7 mm Hg.

FIG. 10B shows a specialized catheter with four side holes that were used in the study described in FIG. 10A to insert into each specimen to take lumen pressure measurements at four different locations (CBD, the Proximal SO, the Body SO, and the Papilla-SO), with the catheter leading to the collection cup, through the outflow catheter and finally into the outflow reservoir.

FIG. 10C shows graphical data related to the changes in lumen pressure and changes in lumen geometry observed in the study described in FIG. 10A as the CBD pressure was gradually increased from 0-17 mm Hg (note here in configuration III how the SO pinches off a pocket of solution, which is then allowed to flow through to the duodenum, a configuration of which is associated with a gradually increasing CBD pressure and decreasing papilla SO pressure (flows presented in FIG. 10C are transient).

FIG. 11A shows the three main regions in which Cholangiocarcinoma (CC) originates, as well as variations of perihiliar CC whose classification is based on the Bismuth scale.

FIG. 11B illustrates how cancer can form in the bile ducts, with tumors beginning to grow within the walls (i), growing along the inner walls (ii and iii), or all of the aforementioned.

FIG. 11C shows a graphical representation of the probability of survival in years after resection for invasive CC versus non-invasive CC (illustrative of the poor mortality rate associated with CC).

FIG. 12 shows a stent according to at least one embodiment hereof.

FIG. 13A shows a schematic of an E. coli bacteria swimming along a flat surface incident to a flowing liquid.

FIG. 13B illustrates various regimes of bacterial swimming trajectories including subpart a) which illustrates regime 1, E. coli bacteria swimming trajectories with respect to a flow direction; subpart b) which illustrates regime 2, E. coli bacteria swimming trajectories with respect to a flow direction with an increased shear rate as compared to that shown in subpart a) of FIG. 13B; subpart c), which illustrates regime 3, E. coli bacteria swimming trajectories with respect to a flow direction, with the trajectory of the E. coli swimming predominantly upstream; and subpart d) which illustrates regime 4, E. coli bacteria swimming trajectories with respect to a flow direction with the highest shear rates out of those shown in FIGS. 13B, subparts a)-d).

FIG. 14 shows a side, cross-sectional view of at least one embodiment of a one-way valve that discourages fluid flow from left to right and encourages fluid flow from right to left as indicated by the arrows.

FIG. 15A shows schematics of a phase transforming cellular material (PXCM) introduced by Restrepo et al. that utilizes a sinusoidal beam snapping mechanism to exhibit solid state energy dissipation and enables the material to transition between stable (or metastable configurations).

FIG. 15B shows 2D models of the functionality of a PXCM material introduced by Zhang et al. that utilizes the same sinusoidal beam in 2 separate designs: the S-type that uses a square-shaped motif (right) and the T-type that utilizes a triangular-shaped motif (left).

FIG. 15C shows photographs of the chiral PXCM studied by Hector et al. that utilizes tape spring ligaments as the segments in a chiral topology to exhibit energy dissipation and phase transformations.

FIG. 15D, top row, is a schematic representative of a bistable architected material analog for a shape memory alloy (ASMA) unit cell made of 2 materials, m₁ and m₂; second row is a plot of E vs T which shows the temperature dependence of Elastic modulus E with temperature T for materials m₁ and m₂; third row are plots of F vs. d and U vs. d, which represent a comparison of the load (F) versus displacement (d) and elastic strain energy (U) versus displacement (d), respectively, for the normal PXCM made of m₁ and the ASMA made of m₁ and m₂ at lower temperatures (note how they are both bistable as indicated by the negative load in the load displacement curve and the potential well in the energy versus displacement plot); and fourth row showing the plots of the third row, but where the studies were done with the temperature increased, which as shown in the plots of the fourth row, lowered the elastic modulus of m₂ in the ASMA (indicative of the ASMA now exhibiting metastable behavior while the PXCM made strictly of m₁ remains bistable). FIG. 15D shows that the elastic modulus m₂ (its stiffness) decreases with increasing temperature while the elastic modulus of m₁ remains relatively constant for the same range of temperature.

FIG. 16A shows a schematic of analog ASMA utilizing PXCMs, more specifically two different materials—one with a temperature independent elastic modulus, m₁ (light grey) and one with a temperature dependent elastic modulus m₂ (black).

FIG. 16B shows the shape memory alloy (SMA) of FIG. 16A at the molecular level, representing the phase transformations that occur in SMAs), where at cool temperatures (e.g., room temperature), the molecules arrange themselves into an unstressed, twinned, martensitic configuration; when the SMA is strained (i.e. pulled apart) significantly enough to induce plastic deformation, the SMA molecules orient themselves into a detwinned, martensitic configuration; when the SMA is unloaded, the SMA has plastified (meaning when all of the load has been removed from the SMA, it has not returned to its original configuration, however, when heat is introduced to the plastically deformed SMA, the molecules reorient themselves into an Austenitic configuration, which allows the SMA to recover elastically and return to its original configuration when unloaded.

FIG. 16C depicts the mechanical behavior exhibit by an ASMA (blue), which is analogous to an SMA transitioning from a twinned martensitic phase to a detwinned martensitic phase and corresponds to the blue stress-strain curve shown in FIG. 16D below; however, when heat is applied to the bistable ASMA, because one of the consisting materials has a temperature dependent young's modulus, it now behaves as a metastable ASMA (red) which corresponds to the red stress-strain curve shown in FIG. 16D.

FIG. 16D shows stress-strain curves representative of how the transformations depicted in FIG. 16B occurs, where there are two stress (sigma) versus strain (epsilon) curves that lie along a temperature, T, axis that comes out of the page, the bottom stress-strain curve corresponding to an ASMA at a low temperature (which behaves similar to an SMA at a low temperature; when load is removed, the stress-strain curve does not return to the origin); but where the temperature is increased, the ASMA transforms from bistable to metastable and returns to the origin of the stress-strain plot observed at a higher temperature (when load is applied and then removed, the ASMA returns to its original configuration).

FIG. 16E shows a diagram illustrating that ASMAs with different wavelengths L can exhibit different temperatures at which they pop from their bistable configuration back to their metastable configuration.

FIG. 16F shows a graph representative of the load-displacement behavior of the same 3 ASMA designs shown in FIG. 16E, where the blue (A), orange (B), and grey (C) curves correspond to the L=50 mm, L=60 mm and L=70 mm ASMA designs, respectively.

FIG. 16G shows a graph representative of the load-displacement behavior of the L=50 mm ASMA cell at 25° C., 30° C., and 38° C., respectively.

FIG. 17A shows graphical data related to the peak stress of an ASMA as a function of the temperature.

FIG. 17B shows a design map of the ASMA featuring the mechanism, Q as a function of the temperature.

FIG. 18A shows a plot graph of load-displacement behavior of an ASMA cell at various temperatures, with A corresponding to the ASMA cell at 25° C., 30° C., and 38° C., respectively. In this case, the ASMA unit cell was heated sufficiently such that the minimum (C curve) surpasses the resistance given by the horizontal dashed line and was thus able to return to its original configuration.

FIG. 18B shows a plot graph displaying F2 values for several temperatures and several ASMA designs. The smaller the ASMA cell wavelengths can achieve large values of F2 and, thus, can do work against larger external resources.

FIGS. 19A-19C show a stent according to at least one embodiment hereof.

FIG. 20A shows a graph of the maximum transition load (F1) of ASMA designs of the present disclosure as a function of temperature. Line A, Line B, Line C, and Line D correspond to the L=35 mm, L=40 mm, L=50 mm, and L=60 mm ASMA cell designs, respectively. This data supports that for each ASMA design presented herein, as the temperature increases, the load required to collapse each cell (F1) decreases, implying that each ASMA cell becomes progressively easier to collapse.

FIG. 20B shows a graph of the minimum load (F2) of ASMA designs of the present disclosure as a function of temperature and supports that each ASMA design works without an external resistance since the F2 value of each design becomes a positive load at a temperature of 45° C.

FIGS. 20C and 20D show plots of data related to the displacement (U2) on each ASMA design, as well as their temperature design as a function of time, with FIG. 20C showing data where each ASMA cell was allowed to rotate and FIG. 20D showing data where the same ASMA cells where they were not allowed to rotate. The data supports that by changing the wavelength of the ASMA cells, they can be designed to exhibit a stagger reversal in displacement with increasing temperature.

FIGS. 21A-21I illustrate the results of three finite element simulations for peristaltic-like motion with ASMA cells, each of which represents one of three stages of this phenomenon. FIG. 21A represents an initial indentation stage in which the acrylic indenter is loaded into the row of ASMA cells strictly in they direction (e.g., to emulate an obstruction becoming clogged in the ASMA stent). FIG. 21B shows a graph of the forces of the ASMA cells of FIG. 21A in the y-direction in the stage of FIG. 21A, which is representative of how the different ASMA cells compete with each other (noting that at 9 seconds in FIG. 21B, L35 has a higher load than either L40 or L50, supporting that L35 is pushing back the hardest on the acrylic indenter), and FIG. 21C shows a graph of the indenter displacement in the x direction during the stage represented in FIG. 21A (no displacement in the x direction for the indenter during this stage was observed). FIG. 21D represents stage 2, which immediately follows the initial indentation stage (stage 1), where an equilibrium simulation was run using a dynamic explicit solver in which the indenter was allowed to move in the x-direction in its indented state. The indenter moved in the positive x direction until an equilibrium position was identified. FIG. 21E shows a graph of the y-reaction forces that each cell exerted on the acrylic indenter during the phase of FIG. 21D (note that L40 had a reaction force that exceeds 400 N), and FIG. 21F shows a graph of the indenter displacement in the x direction during the stage represented in FIG. 21D (data supporting the indenter found an equilibrium position as the x-indenter displacement plateaus around 13.1 mm). Note at stage 2, Rayleigh damping was used to locate this particular equilibrium point. FIG. 21G represents a final stage (stage 3) involved in activating the ASMA cells with temperature changes (e.g., temperature increased from 0° C. to 10° C., with FIG. 21H showing a graph of the forces of the ASMA cells in the stage of FIG. 21G and FIG. 21I showing a graph of the indenter displacement in the x direction during the same final stage.

FIG. 22 shows a graph of the stage 3 results involving two 45° C. temperature cycles (blue curve), with the second cycle indicated in red.

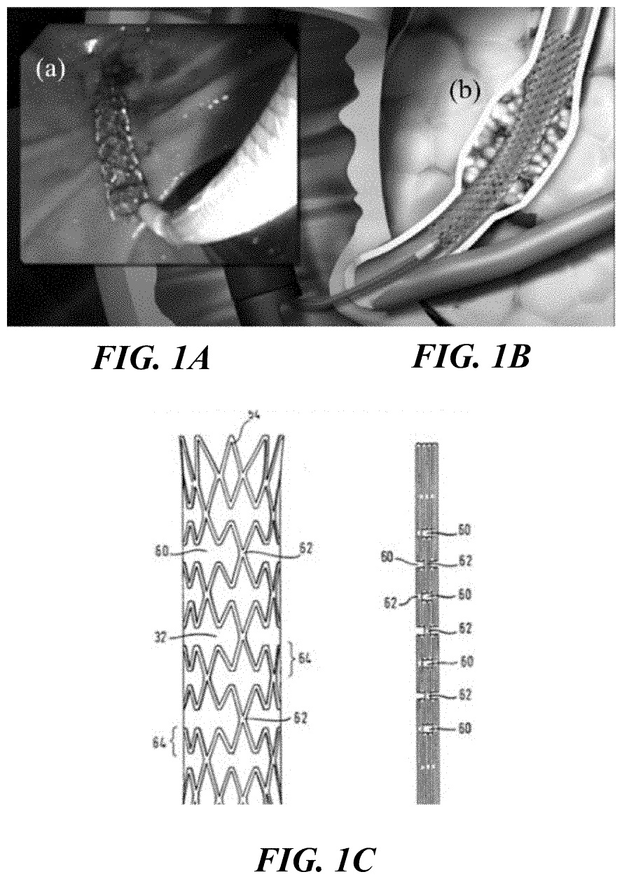

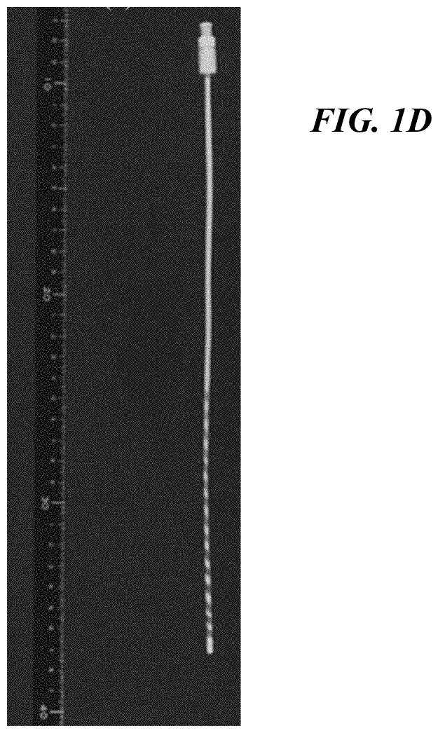

While the present disclosure is susceptible to various modifications and alternative forms, exemplary embodiments thereof are shown by way of example in the drawings and are herein described in detail.

### DETAILED DESCRIPTION

For the purposes of promoting an understanding of the principles of the present disclosure, reference will now be made to the embodiments illustrated in the drawings and specific language will be used to describe the same. It will nevertheless be understood that no limitation of scope is intended by the description of these embodiments. On the contrary, this disclosure is intended to cover alternatives, modifications, and equivalents as may be included within the spirit and scope of this application as defined by the appended claims. As previously noted, while this technology may be illustrated and described in one or more preferred embodiments, the stents and methods hereof may comprise many different configurations, forms, materials, and accessories.

Stents and methods of treating a subject having a wholly or partially compressed or obstructed duct are provided. The stents hereof comprise phase transforming cellular materials (PXCM) and are configured to open and close with the frequency of the Sphincter of Oddi (SO), for example, as a result of either a pressure gradient or in response to cholecystokinin (CKK) levels. In at least one embodiment, a stent is provided that is an artificial, one-way valve that can replace a dysfunctional (or preserve the functionality of a) SO. The stents and methods hereof provide significant benefits over existing stent technologies, especially as a means for allowing cancer treatments to continue unimpeded while a stent is implanted or otherwise placed within the subject. In certain embodiments, the stent exhibits controllable motion analogous to peristalsis as seen in the esophagus and the intestines, which allows for the maintenance of bile flow while also guarding against harmful bacterial infection. Peristaltic motion is induced when an object (i.e. a food bolus in the esophagus, for example) is subjected to a traveling contraction wave that does work on that object to push it forward against resistive forces (e.g., closure forces ahead of the food bolus in the esophagus). In certain embodiments, the stents hereof can move an external resistance along at least a portion of the length of the stent (e.g., a tube).

A background on the biliary tree, its component parts, and the various considerations that affect flow therethrough is provided to facilitate understanding of the principles of operation that underly the stents and methods provided herein.

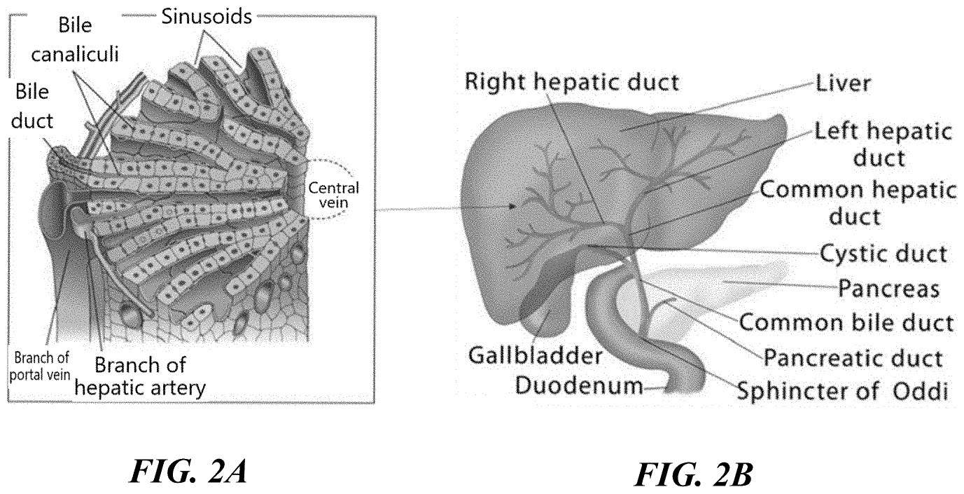

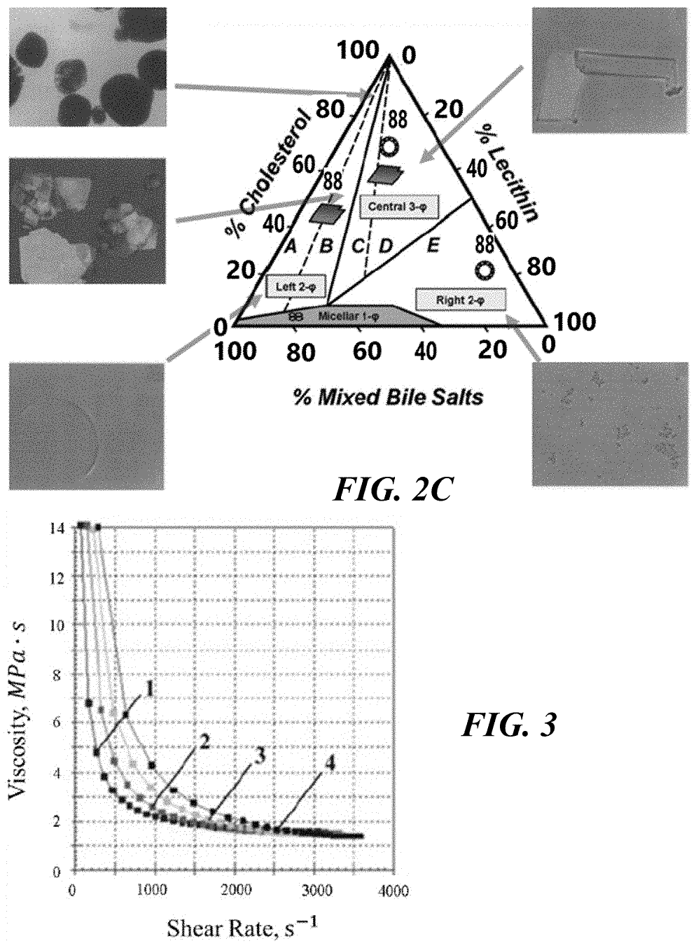

The biliary tree is a component of the gastrointestinal tract of any organism with a gall bladder and consists of a series of organs and ducts that are tasked with the creation, storage, transportation, and release of bile to the duodenum, which sequentially leads to the small intestines. As shown in FIG. 2B, the biliary tree consists of the liver, the left and right hepatic ducts, the common hepatic duct, the cystic duct, the gall bladder, the common bile duct (CBD), and the SO, all of which necessitate the transportation of bile.

### The Liver

The liver is a large organ in the body that is oriented in the mid-right section of a subject's torso. It is in the liver that a fluid called bile is created and secreted into the “upper branches” of the intrahepatic bile ducts that extend through the liver (see FIG. 2B). The liver produces approximately 0.5-1 liter of bile every day in a healthy human being and is one of two major sources of pressure in the biliary system (the other source being the gall bladder). In particular, a type of cell known as a hepatocyte (Bile Canaliculi) lines the intrahepatic bile ducts and is specifically responsible for the secretion of bile (see FIG. 2A).

In healthy patients, bile is a Newtonian fluid (non-pathological, shear rate-independent viscosity) that aids in the digestion of fats. The viscosity of bile ranges between 1-10 mPa·s and has a density of approximately 1000 kg/m³. The Reynolds number for bile depends on its viscosity as well as the diameter of the particular duct through which it flows and varies from subject to subject with values reported between 1-40. The flow rate of healthy (Newtonian) bile can be modeled via the following:

$$Q = a \frac{π {D}^{4}}{128 µ} {( \frac{Δ p}{L} )}^{b} \qquad ( 1 )$$

where Q is the flow rate, D is the diameter of the duct through which the bile is flowing, μ is the viscosity of bile, L is the length of the duct, Δp is the pressure difference between the inlet and outlet of the duct, and a and b are empirical constants that depend upon the particular duct through which the bile is flowing.

Bile is continuously secreted in the liver all day long and stored in the gall bladder until it is needed. The rheological properties of bile depend on several factors including but not limited to age, occupation, diet, and bacterial content. It is commonly accepted (and well supported in the literature) that pathological bile (e.g., bile infected with harmful bacteria) behaves as a Non-Newtonian fluid (i.e. shear rate dependent viscosity), such as in subjects diagnosed with cholelithiasis.

Supporting this, FIG. 3 illustrates the shear-rate dependency of four subjects diagnosed with jaundice brought on by cholelithiasis (gall stones). Bile specimens were acquired from each specimen and the shear rate dependency of each sample was obtained experimentally via a viscometer. For a large range of shear rates (˜10-3000 s⁻¹), each bile sample exhibited non-Newtonian (shear rate-dependent viscosity), and at very large shear rates (˜>3000 s⁻¹) all samples reverted to exhibiting Newtonian behavior (shear rate-independent viscosity). However, these large shear rates are not representative of normal physiological function.

Further, the presence of biliary sludge (Non-Newtonian bile) in the biliary tract also implies a major imbalance of the following three critical components of bile: cholesterol, phospholipids, and bile salts. Bile salts consist of both hydrophobic and hydrophilic components and can form micelles in the bile which are used to dissolve cholesterol. However, if there is a plethora of cholesterol present in bile, it can bond with the phospholipid component of bile instead to form vesicles, which can fuse together to form biliary sludge (e.g., liquid crystals) or gallstones. FIG. 2C displays the different physical phases of bile depending on the ratios between cholesterol, phospholipids, and bile salts (each phase labeled A-E). Each phase of bile is primarily a function of bile salt to phospholipid ratio and the cholesterol saturation index (CSI). The phospholipid content is the lowest in Region A where arc-like crystals form. Region B is characterized by high phospholipid concentrations where monohydrate crystals form. Region C comprises typical phospholipid content and can form biliary sludge. Region D comprises of moderate to low phospholipid content, starts as biliary sludge and nucleates to form monohydrate crystals. Region E can have high phospholipid concentrations and form only biliary sludge (no solid crystals are typically formed in this phase of bile).

Accordingly, stent design should take into account the maintenance of bile flow in the presence of a bacterial infection that manipulates the physical phase of bile and its rheological properties.

### The Hepatic Ducts

As shown in FIG. 2B, the hepatic ducts comprise the “upper part” of the biliary tree in most vertebrates and, as part of the liver, are tasked with the creation and secretion of bile. Each of these ducts converge on either the left hepatic duct or the right hepatic duct, which also both converge together to form the common hepatic duct. The common hepatic duct serves as the main conduit that necessitates the transportation of bile out of the liver. This transportation is associated with a pressure difference between the liver (˜2941 Pa) and the common hepatic and common bile ducts (˜490-980 Pa). Perhaps more specifically, this pressure gradient is the difference in the intraductal pressure (measured relative to the pressure outside of the subject's body) between the intrahepatic ducts in the liver and the pressure of the common hepatic and common bile ducts.

### The Cystic Duct

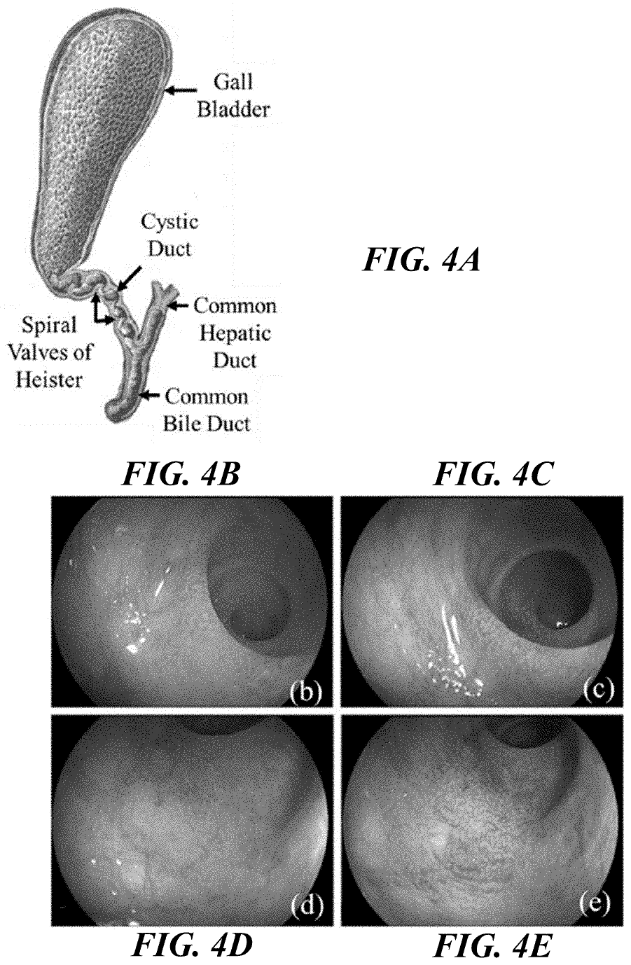

As illustrated in FIG. 4A, after leaving the common hepatic duct, bile can flow through either the CBD and out into the duodenum, or it can flow through the cystic duct for storage in the gall bladder. The cystic duct joins the common hepatic duct to the CBD and connects this intersection to the gall bladder (see FIG. 2B). The mean diameter of the cystic duct varies (e.g., between 2-5 mm in a human subject) and can also vary in length (e.g., between 4-65 mm long in a human subject).

The cystic duct has a complex intraductal geometry that comprises the valves of Heister (see FIG. 4A) and can consist of between 2-14 folds (i.e. spirals or folds). Interestingly, intersubject variations in this complex geometry can greatly contribute to a subject's susceptibility to developing cholelithiasis.

The cystic duct (and, more specifically, at least the folds within the Valves of Heister) acts as a passive flow resistor to control bile flow out of the gall bladder. The two most important geometrical parameters that are responsible for increasing the flow resistance within the cystic duct are the baffle clearance (c/D) (i.e. lumen size) and the number of baffles therein (n) (number of folds in the valves of Heister). The least significant geometrical parameters affecting flow resistance are the overall curvature of the cystic duct and the angle between the neck and the gall bladder. The flow resistance (R) is generally given as the ratio of the pressure drop (Δp) across the cystic duct to its flow rate (Q), however, to be compared with the Reynolds number.

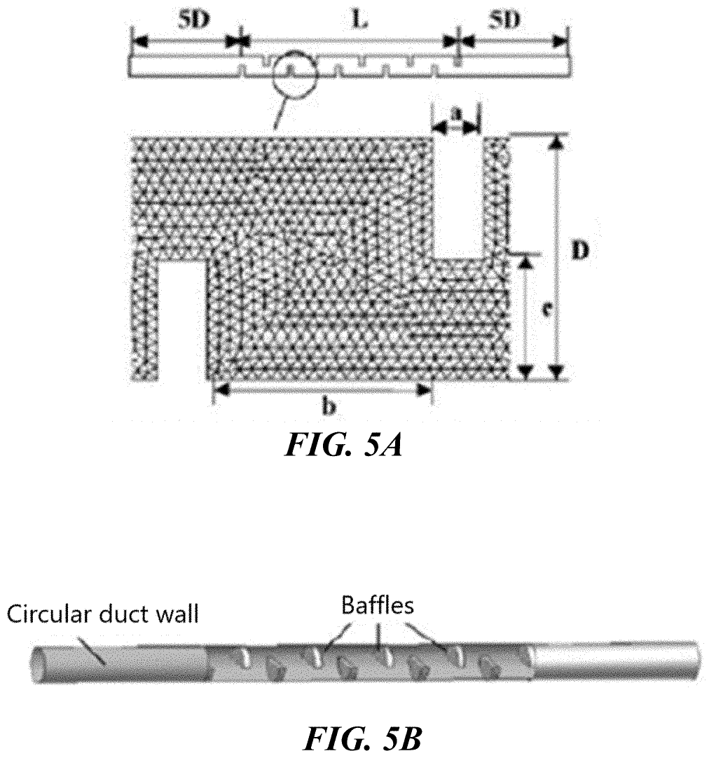

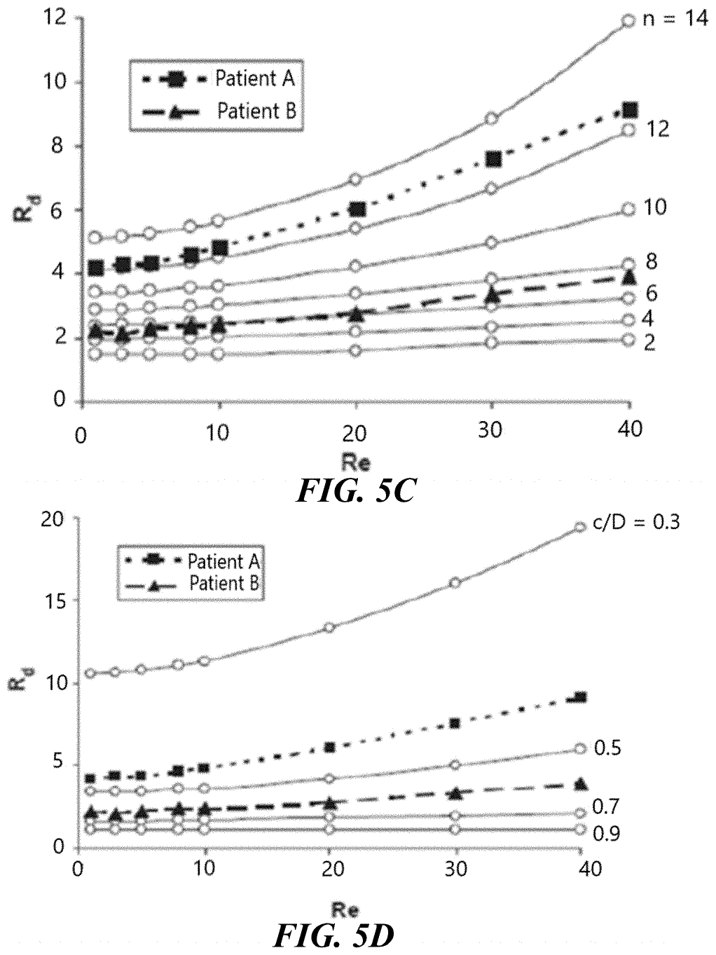

FIGS. 5C and 5D show data measured from 2D and 3D models developed (shown in FIGS. 5A and 5B, respectively) of the complex lumen geometry of the cystic duct to observe how the flow resistance is affected by the luminal geometry and the Reynolds number (i.e. Reynolds number and baffle numbers are independent variables controlled to see how the flow resistance changes). As shown in FIGS. 5C and 5D, an increase in the normalized flow resistance was observed that correlated with an increased number of baffles and Reynolds number (R_{d}=Rₙ/R₀, where Rₙ is the resistance through a model with n baffles and R₀ is the resistance through a model with 0 baffles for normalization purposes). The data supports that R_{d} increases when the flow is dominated by inertial forces with a characteristically high Reynolds number (e.g., ˜>10), while for lower Reynolds number flows (e.g., ˜<10; dominated by viscous forces) the flow resistance tends to decrease. Accordingly, the relatively higher flow resistance in the cystic duct indicates that the gall bladder has to exert a relatively larger force to expel bile into the cystic duct.

Since the cystic duct is a bidirectional conduit, the pressure difference across it can not only dictate the flow rate, but also the direction of the flow. It has been shown that the pressure required to initiate bile flow through the cystic duct ranges between 0.1-8 cm H₂0 (˜9.8-784.5 Pa). This large variation is in part attributable to the wide variety of geometrical variations exhibited by the cystic duct between subjects.

### The Gall Bladder

Moving down the system, the gall bladder is the only dynamic organ in the biliary system and can act as both a storage unit for bile and a pressure reservoir/regulator. There can be a synchronized cooperation between the SO and the gall bladder that transfers bile from the gall bladder to the duodenum. This synchronized cooperation between the gall bladder and the SO can be analogously referred to as a “Pump-Pipe” system and is driven by a neural-hormonal-mechanically coupled mechanism. For example, during digestion, a hormone known as cholecystokinin (CCK) is released into the blood stream by the endocrine system. CCK can stimulate both a contraction in the gall bladder and a relaxation in the SO, which creates a pressure gradient between the gall bladder and SO in favor of bile flowing into the duodenum.

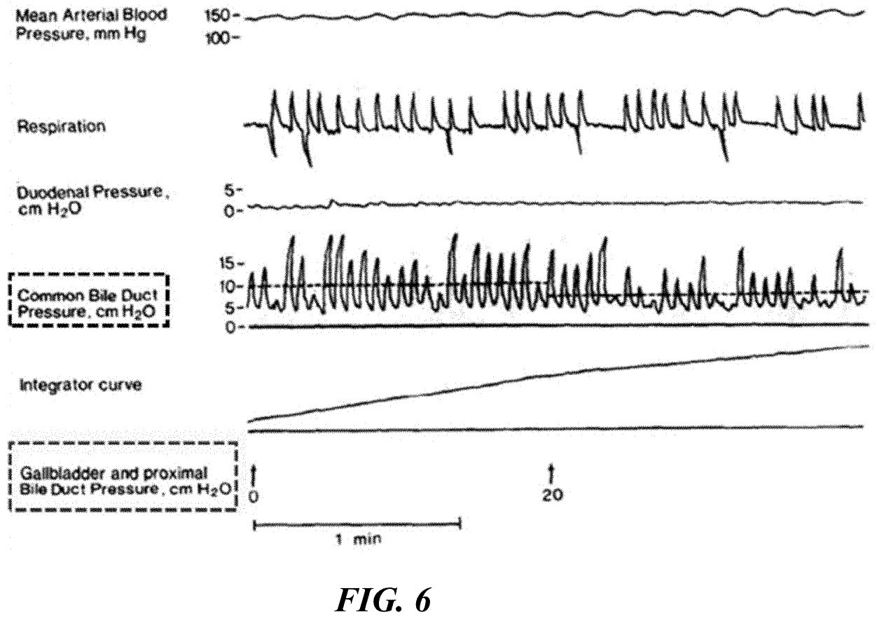

FIG. 6 shows data from a series of intraductal pressure readings taken in a study designed to assess the pressure gradient between the gall bladder and SO during physiological conditions. As shown in FIG. 6, there is a decrease in the base line pressure of the distal SO (termed the “Common Bile Duct” in FIG. 6) that correlates with an increase in pressure within the gall bladder. This cooperation either drives bile into the gall bladder for storage or, in the cases represented in FIG. 6, out of the gall bladder, down the CBD, and out into the duodenum through the SO.

The human gall bladder has a resting pressure ranging between 10-20 cm H₂0 (˜980.6-1961.3 Pa). After a meal, in response to CCK released into the bloodstream, the pressure in the gall bladder increases to a range between 26.2-38.7 cm H₂0 (˜2569.3-3795.1 Pa). The human gall bladder empties at an average rate of 1 mL/minute with a maximum flow rate of ˜5 mL/minute as suggested via ultrasonographic imaging (not shown).

To understand the pressure gradient that drives the motor function/cooperation between the gall bladder and the SO in a healthy body, the fluid mechanics of bile involved with the filling and emptying of the gall bladder and the flow of bile through the cystic duct must be considered. The gall bladder is essentially a hollow sac that is approximately 7-10 cm long and 3-4 cm wide in human adults. The average storage capacity of a healthy gall bladder ranges between 20-30 mL, however, the total occupying volume can be dependent upon the pressure within the gall bladder and the compliance (e.g., stiffness) of the walls constituting the gall bladder. Additionally, motor function of the gall bladder can be related to the pressure drop between the gall bladder and the SO (see FIG. 6), the flow rate of bile out of the gall bladder, and the flow resistance of bile. To expand on this, the flow rate (Q), the flow resistance (R) and the pressure drop (Δp) are also related to the lumen geometry of the gall bladder, the cystic duct, the CBD, and the SO.

The relationship between the pressure drop (Δp) and the flow rate (Q) between the gall bladder and the SO has been loosely modeled with the following expression:

Δpⁿ∝Q (2)

where n varies between 1.47-2.05 and depends explicitly on the location of interest from the gall bladder.

Heretofore, mechanical studies of gall bladder motor function have concentrated on the following “constitutive relationships”: (1) gall bladder volume-vs-pressure drop; and (2) length-vs-tension of a strip of gall bladder muscle. Volume-vs-pressure is more commonly studied and accepted, while the constitutive relations governed by the length-vs-tension of a strip of gall bladder muscle are not as well understood or accepted in literature. The relationship between the volume of bile (V) and the pressure (p) in the gall bladder has been modeled simply with the following formula:

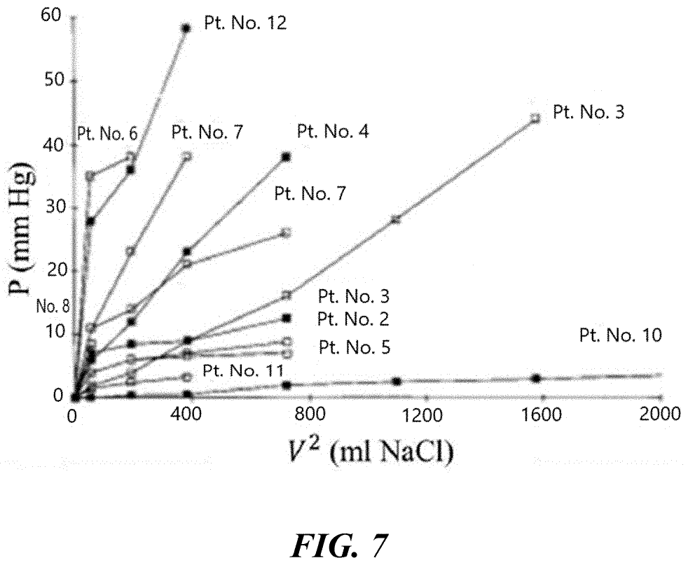

CV²=p (3)

where C is the compliance of the gall bladder. It has been shown, experimentally in opossums, that the compliance (and thus the pressure) drops when CCK is infused into the body. See, e.g., Ryan and Cohen, Gallbladder pressure-volume response to gastrointestinal hormones, Am J Physiol. 1976, 230: 1461-1465. The compliance of the gall bladder (see FIG. 7) will vary from subject to subject due to anatomical variations, most of which occur in the cystic duct; however, in general there is a linear relationship with some degree of error.

### The Common Bile Duct (CBD)

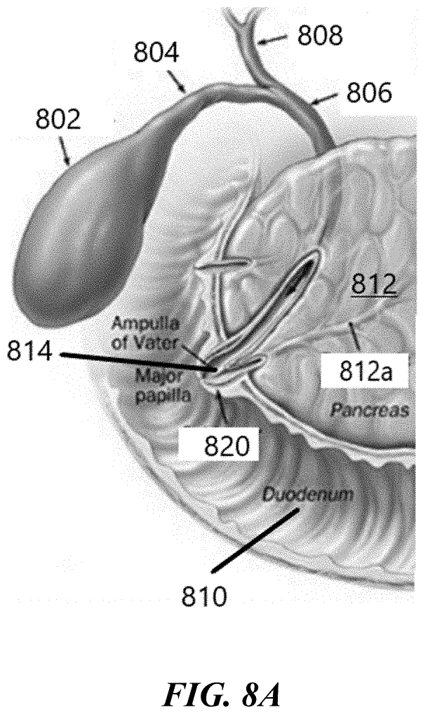

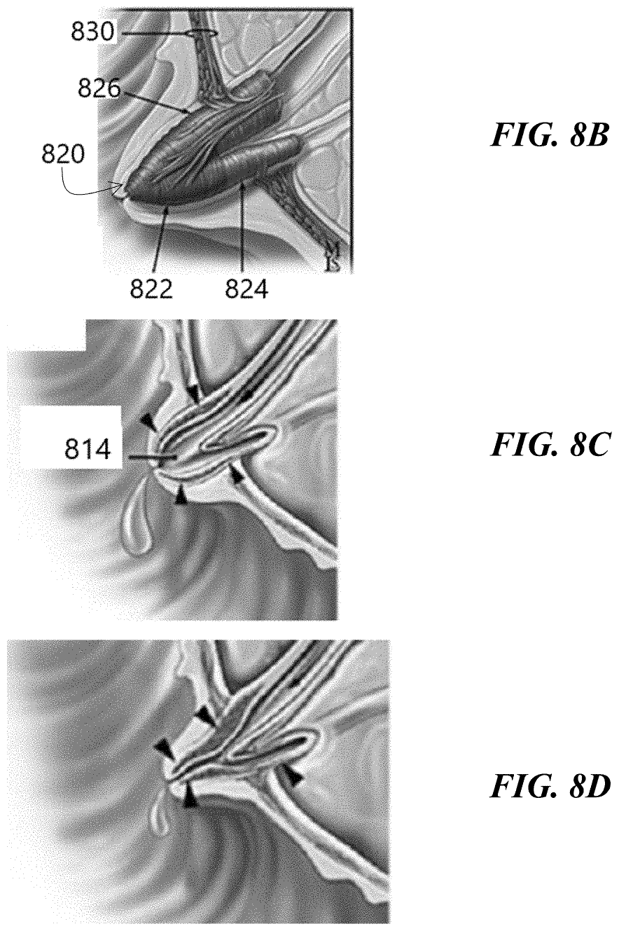

Now referring to FIG. 8A, when bile is needed for digestion, it is diverted out of the gall bladder 802, through the cystic duct 804, and down through the CBD 806 (common hepatic duct identified as 808 for reference in FIG. 8A). The CBD 806 is the main conduit through which bile travels to exit to the duodenum 810. Most of the time, the CBD 806 is merely covered by pancreatic tissue in some areas, however, it has been observed to be embedded completely within the pancreas 812. The CBD 806 has an external diameter that remains fairly constant along its length but can have some degree of variation between human subjects (e.g., between 5-13 mm). The inner diameter of the CBD 806 typically tapers from the T-junction connecting it to the cystic and common hepatic ducts 804, 808 (respectively), to the ampulla 814. The inner diameter can start (i.e. at the T-junction) as large as 4-12.5 mm and shrink to anywhere at or between 1.5-7.5 mm. The CBD 806 joins up with the pancreatic duct 812a at a confluence known as the ampulla of Vater 814 and it is here that these two ducts become surrounded by a small group of muscle known as the Sphincter of Oddi 820 (identified herein as SO or SO 820) (see FIGS. 8A-8D).

The SO 820 is a group of muscles tasked with the following primary functions: (1) regulating the flow of bile and pancreatic fluid into the duodenum 810; (2) diverting the flow of hepatic bile into the gall bladder 802 for storage; and (3) preventing the flow of duodenal contents up through the pancreatic ducts 812a and the biliary tree. When a fatty meal is ingested, the body produces CCK which, as previously described, causes the gall bladder 802 to contract. This squeezes bile into the cystic bile duct 804 (see FIG. 8A), simultaneously relaxes the SO 820, and allows bile to flow through to the duodenum 810.

The SO consists of both circular and longitudinal smooth muscle fibers, which can be discretized into three main regions. As illustrated in FIG. 8B, the regions of the SO include the sphincter papillae 822 (the portion of the SO that protrudes approximately 1 cm into the duodenum), the sphincter pancreaticus 824 (which covers the end of the pancreatic duct 812a (5-6 mm)), and the sphincter choledochus 826 (which covers the end of the CBD (5-6 mm)). Duodenal musculature is identified as 830 in FIG. 8B. The normal appearance of the SO 820 via endoscopy includes only the sphincter papillae 822, which consists of the major papilla, which has about a 1 mm orifice diameter.

When a meal is ingested, it is this group of muscles that relax and contract and ultimately change the stiffness of the entire SO 820 (see FIGS. 8C and 8D). As noted above, the SO 820 primarily surrounds the confluence of the CBD 806 and the pancreatic duct 812a, which is an opening known as the ampulla of Vater 814. This region can be an area of varying pressure, which acts to either divert bile to the gall bladder 802 (at high pressure) or allow it to flow through to the duodenum 810 at lower pressure. For reference, FIG. 8C shows the shape of the ampulla of Vater 814 when the SO 820 is open (i.e. the muscles of the SO 820 are compliant and allow for the flow of bile into the duodenum 810). Similarly, FIG. 8D shows the shape of the ampulla of Vater 814 when the SO 820 is closed (i.e. the muscles of the SO 820 are rigid/contracted and do not allow for the flow of bile into the duodenum 810—or the backflow of bacteria into the CBD 806).

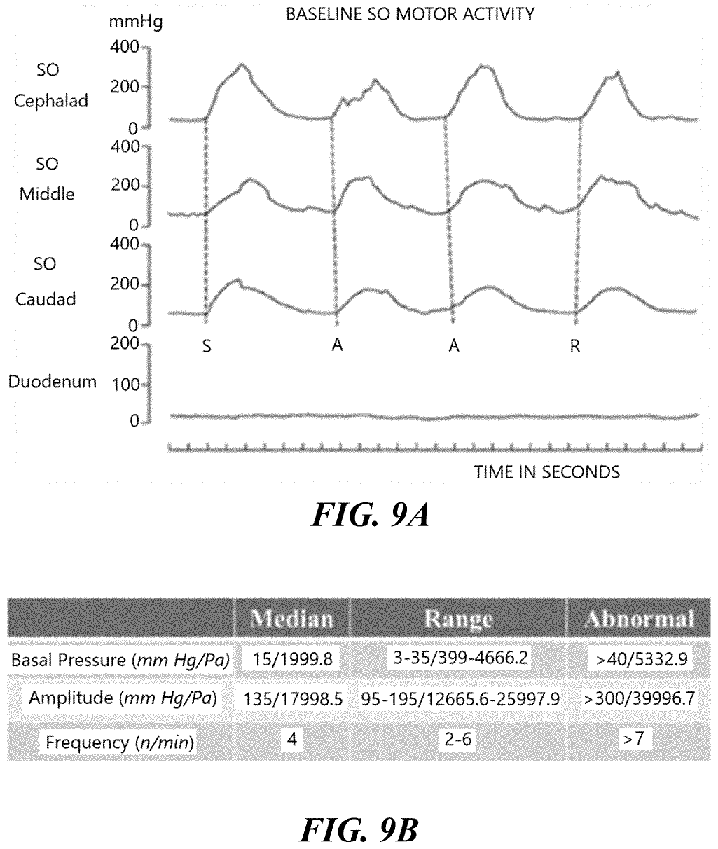

As shown in FIG. 9A, superimposed on the resting pressure of the SO 820 are rhythmic spikes in pressure ranging between 50-150 mm Hg (˜6666.1-19998.4 Pa) and occur at a frequency of 2-5 spikes per minute. The pressure of the duodenum 810 is also shown for reference. FIG. 9B shows a table of data relating to healthy and abnormal pressure ranges within the SO during these rhythmic spikes.

The frequency with which the SO phastically opens and closes depends on whether the gall bladder is filling or emptying. When the gall bladder is empty, the intraductal pressure in the ampulla of Vater (surrounded by a constricted SO) can be as much as 3 times larger than the pressure in the empty gall bladder. Accordingly, bile is diverted from flowing down the common bile duct to the ampulla of Vater and through the cystic duct into the gall bladder for storage. After a spike has occurred, CCK lowers the pressure of the SO, which opens the SO and allows bile to flow through the SO and into the duodenum. These contractions have also been shown to keep the SO opening free of liquid or solidified bile.

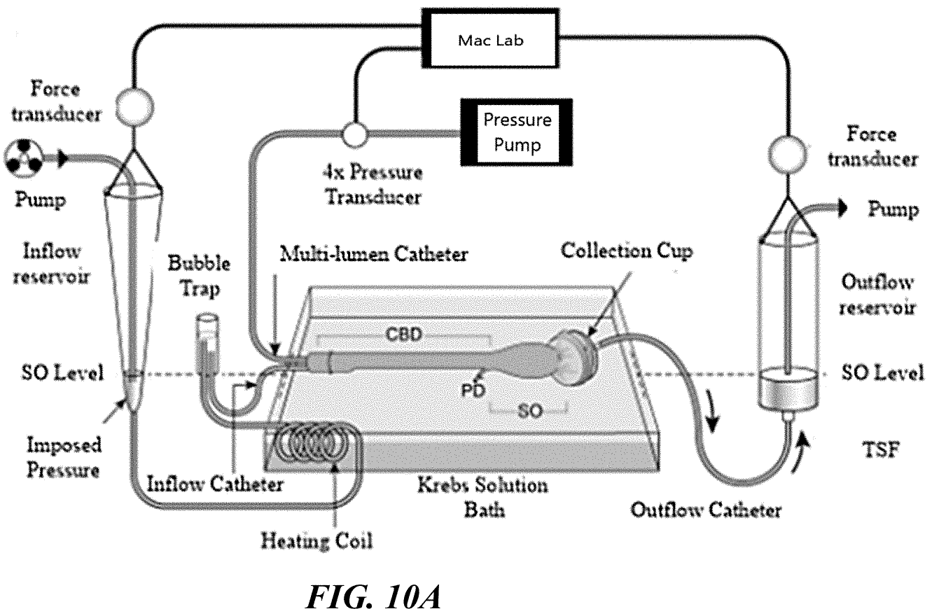

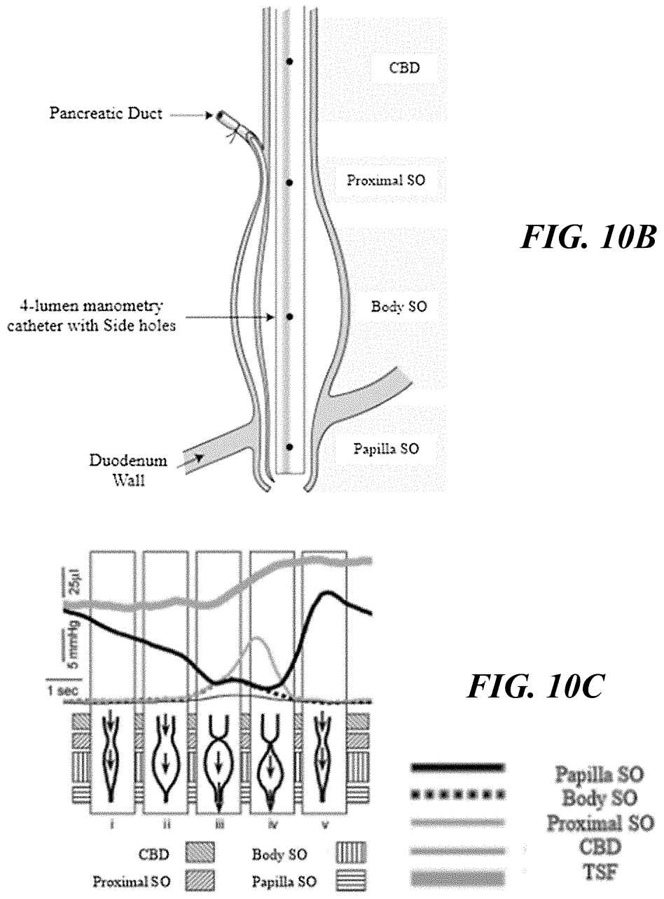

While these phastic pressure changes in the SO are well reported in the literature, very little has been reported on the geometrical changes in the SO and how it can act as a resistor to bile flow. See, e.g., Thune et al., Reflex regulation of flow resistance in the feline sphincter of Oddi by hydrostatic pressure in the biliary tract, Gastroenterology 1986, 91(6): 1364-1369; Otto et al., A comparison of resistances to flow through the cystic duct and the sphincter of Oddi, J Surg Res. 1979, 27:68-72; Toouli et al., Motor function of the opossum sphincter of Oddi, J Clin Investigation 1983, 71(2): 208-220; Guelrud et al., Sphincter of Oddi manometry in healthy volunteers, Digestive Diseases & Sci 1990, 35(1): 38-46. One work (on Australian Possums) sought to understand the mechanics of the SO by removing the CBD (to the hepatic duct junction), pancreatic tissue, SO and 4 cm of attached duodenum in toto (thus eliminating any neural or hormonal stimuli) and placing these ducts into a modified Krebs-Henseleit buffer (Kreb's solution). See Grivell et al., The possum sphincter of Oddi pumps or resists flow depending on common bile duct pressure: a multilumen manometry study, J of Phys 2004, 558(2): 611-622. Kreb's solution was developed in the early 1900s and consists of potassium, sodium, magnesium, calcium, chloride, and phosphates. This solution is similar to that of extracellular fluid and is especially important for studies involving muscle contractions since these contractions are typically dependent upon ion gradients. FIG. 10A illustrates the experimental apparatus used in this study for observing SO motor function for each possum specimen.

By removing the biliary tree from the possums, any behavior exhibited by the SO (ampulla region) was purely mechanical and not due to neural or hormonal stimuli. The natural pressure in the hepatic and common bile ducts were stimulated as well as the duodenum via an inflow and outflow pump. The imposed CBD pressure was manipulated by modifying the height of the inflow reservoir, via a pump to 17 mmHg. SO motility (i.e. phastic pressure contractions) was recorded via cannulation of the CBD with a four-lumen pico-manometry catheter (see FIG. 10B). This catheter was calibrated such that when placed external to but at the same height as the SO, it gave a recording of 0 mm Hg. Imposed CBD pressure was increased continuously from 0-17 mmHg. In separate experiments, the imposed duodenal pressure was also increased from 0 to 4- or 7-mm Hg respectively, by elevating the outflow reservoir 5 or 10 cm respectively. When Krebs solution was allowed to flow through the ducts from the common hepatic duct to the SO, the data shown in FIG. 10C was observed related to the geometrical and mechanical response of the SO.

When the CBD pressure was gradually increased, the SO exhibited 4 distinct geometric configurations (see FIG. 10C, configurations i-iv) before returning to its original configuration (FIG. 10C, configuration v). It was observed that as the CBD pressure increased (FIG. 10C, configurations i and ii), Krebs solution was allowed to fill the Body-SO which acted as a pocket. In configuration iii, the Proximal SO pinched off access to the Body SO, which restricted more Krebs solution from entering. This decreased the pressure in the Papilla SO, allowing Krebs solution to flow through to the collection cup (see FIG. 10A) and to the outflow reservoir (FIG. 10C, configurations iii and iv). These geometric changes in response to incident pressure and fluid are crucial to the SO's primary functions, especially with respect to hindering the reflux of duodenal contents into the biliary tree.

### Surface Tension

Another mechanism that plays a role in the protection of the biliary and pancreatic systems is bile surface tension. Surface tension controls the extent to which a fluid will wet a surface or flow through an orifice under an applied pressure. This phenomenon is driven via two mechanisms. The first is the existence of an inward force that is exerted on the liquid molecules at the surface, which causes the liquid at the surface to shrink. The second is a tangential force that is exerted on the liquid at the surface. In thinking about bile passage through the SO, a simple thought experiment is appropriate: what is the maximum hole size that can prevent a liquid from passing through a hole, similar to the major papilla in the SO? In the case of a quiescent liquid, the pressure exerted by the liquid on a hole must exceed that of the Laplace Pressure. The Laplace Pressure exerted by a body of liquid is given via Eq. 4:

$${δ}_{p} = γ \cdot ( \frac{1}{{R}_{x}} + \frac{1}{{R}_{y}} ) \qquad ( 4 )$$

where, γ is fluid surface tension, and Rₓ and R_{y} are the radii of the water droplet interacting with the hole of diameter, d. When the pressure, p=ρgh, surpasses that of the Laplace Pressure (Eq. 4), the liquid will flow through the hole. Note that ρ is the mass density of the liquid, g is the acceleration due to gravity, and h is the height of the body of liquid above the surface with the hole. Thus, in addition to a pressure gradient, pressure driven bile flow defeats the surface tension effect at the major papilla.

### Bile Duct Cancer and Stent Considerations

Bile duct cancer, also known as Cholangiocarcinoma (CC), occurs when atypical cells grow out of control inside of the biliary tree. This type of cancer can be classified based upon where it originates; intrahepatic originates in the hepatic bile ducts that branch through the liver and extrahepatic originates outside of the liver in the CBD, the SO or in the cystic ducts. Cholangiocarcinoma accounts for 10-15% of all hepatobiliary malignancies, and mostly arises within the extrahepatic ducts (i.e. the cystic duct, CBD, and/or in the ampulla of Vater/major papilla). Generally, CC progresses insidiously, is difficult to diagnose and a has extremely poor prognosis and mortality rate. Effective surgery to remove such cancers often fail due to characteristically late clinical presentation of these tumors. CC typically has a survival rate between 3-6 months. Additionally, and importantly, in addition to its rapid growth and late diagnosis, CC is difficult to treat due to the wide anatomical variations that can be observed in different subjects.

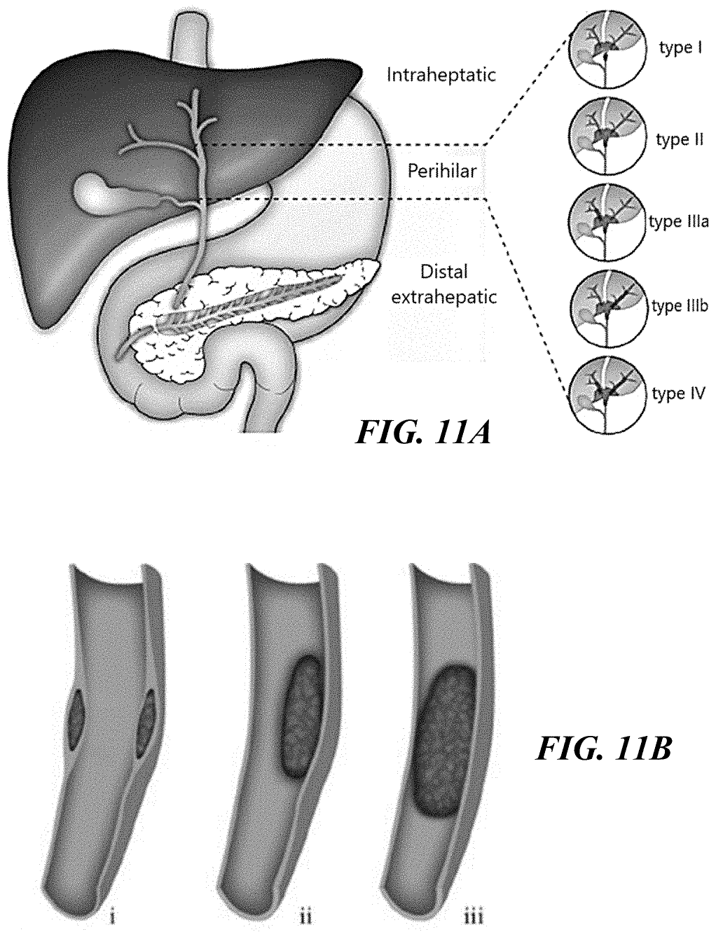

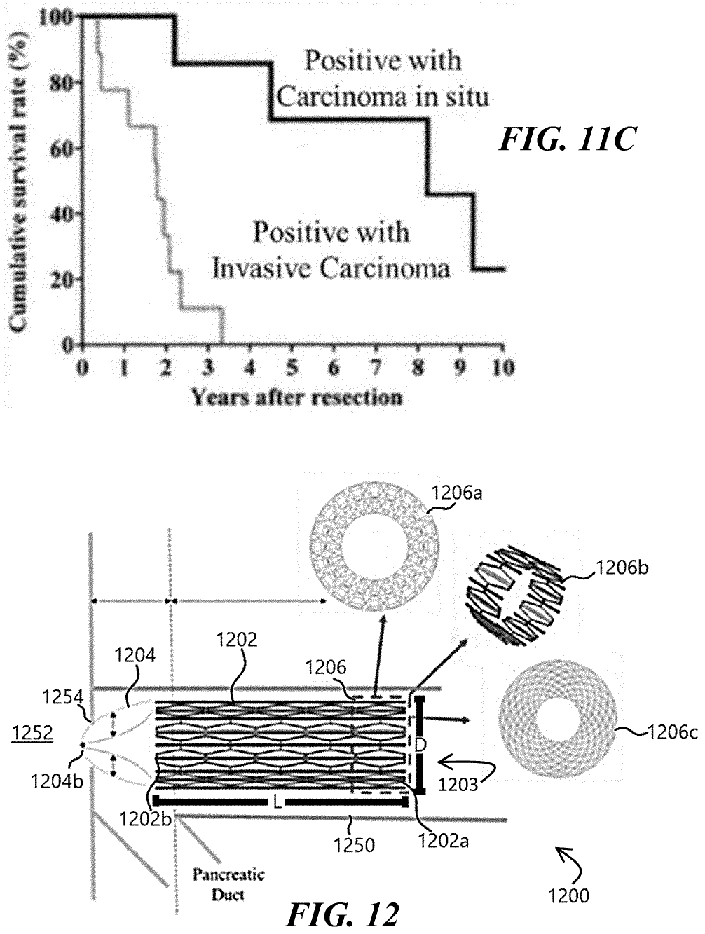

While the discussion presented herein may, at times, focus on extrahepatic CC, there are many types of intrahepatic (hiliar) CC (based upon the Bismuth scale) to which the devices and methods hereof are equally applicable. FIG. 11A illustrates different variations of CC, all of which have a poor prognosis and high mortality rate, FIG. 11B illustrates how cancer can form in the bile ducts, and FIG. 11C shows a graphical representation of the poor mortality rate associated with CC.

Now referring to FIG. 12, at least one embodiment of a stent 1200 is shown. The stent 1200 comprises a first region 1202 and a second region 1204 aligned along a longitudinal axis of the stent 1200. While the first region 1202 operates to support a constricted or otherwise impeded targeted lumen, the second region 1204 is configured for placement within an opening adjacent to the targeted lumen. In at least one exemplary embodiment where the stent 1200 is used to treat bile duct cancer, the targeted lumen comprises CBD 1250 (i.e. that is constricted by a tumor or otherwise obstructed (e.g., wholly or partially)) and the opening adjacent to the targeted lumen is the ampulla of Vater 1254 as shown in FIG. 12.

The first section 1202 can be more radially stiff than the second section 1204, but the second section 1204 is capable of emulating the mechanics and associated geometric changes of the opening (e.g., the ampulla of Vater 1254 when the SO muscles contract and relax). Accordingly, the stent 1200 can not only counteract force caused by the presence of a tumor or other obstruction to facilitate flow through the targeted/obstructed lumen (i.e. via first region 1202), but also allow for a functioning valve (i.e. ampulla of Vater) to prevent harmful backflow through the system (i.e. via second region 1204). While the biliary system and specifically the CBD, ampulla of Vater, and the SO are described herein, the present stents 1200 and methods of treatment using the same can be applied to other lumens and ducts as will be evident to one of skill in the art.

In certain embodiments, the first region 1202 comprises an upstream end 1202a, a downstream end 1202b, and a lumen 1203 extending a length L between the upstream end 1202a and the downstream end 1202b. The first region 1202 can comprise an elongated tubular configuration where each of the downstream and upstream ends 1202a, 1202b are expanded radially such that the first region 1202 defines a first diameter D along the length of the lumen 1203.

The second region 1204 can be formed out of one or more types of architected materials, including without limitation, phase transforming cellular materials (PXCMs) and/or artificial shape memory alloys (ASMAs), as described in additional detail below (although other materials can be incorporated as desired). The second region 1204 is coupled with the downstream end 1202b of the first region 1202 and defines an outlet 1204b that is in fluid communication with the lumen 1203 of the first region 1202. The outlet 1204b of the second region 1204 is configured to open and close in a pulsating fashion similar to the SO's motorized behavior that is graphically shown and otherwise described in connection with FIGS. 6, 9A, 9B, 10A, and 10B. In operation, fluid (e.g., bile) flows through the lumen 1203 of the first region 1202 and, when the outlet 1204b is in its open configuration, the fluid flows therethrough and into the duodenum 1252.

As described in additional detail below, the pulsating behavior of the second region 1204 stems from the PXCMs and/or ASMAs employed and can be timed and/or regulated via any one of the following stimuli: (1) unstable behavior in the compliant sinusoidal mechanisms; (2) temperature changes (e.g., where the stent 1200 comprises one or more ASMAs as described below); (3) changes in pressure due to incident bile; (4) changes in the local concentration of CCK; (5) application of external resistance; and (6) self-actuation of PXCM (e.g., where the stent 1200 comprises one or more PXCMs as described below). In at least one exemplary embodiment (for example, where the stent 1200 comprises a biliary stent), the second region 1204 is configured for placement within the ampulla of Vater 1254 of a subject and is capable of emulating the mechanics and associated geometric changes of the ampulla of Vater 1254 when the SO muscles contract and relax.

The first region 1202 of the stent 1200 is for placement within a targeted lumen and operates to maintain normal fluid flow therethrough. The first region 1202 can be any tubular stent (or portion thereof) known in the art that is appropriate for placement within a biological lumen and capable of providing radial expansion and scaffolding within the targeted lumen (e.g., despite an obstruction or constriction) to improve and/or maintain flow therethrough. For example, and without limitation, the first region 1202 can be formed of a plastic or a woven metal mesh. The first region 1202 can also be composed of a base material that is hydrophilic or ionizing such that the first region 1202 carries a negative charge to deter bacterial colonization. The first region 1202 can also comprise one or more types of PXCMs and/or ASMAs.

Where desired, at least the first region 1202 can be a self-expanding stent to facilitate installation within the CBD 1250 (or other targeted lumen to which it is applied). Accordingly, in addition to the elongated tubular configuration, the first region 1202 further comprises a reduced configuration where each of the downstream and upstream ends 1202a, 1202b are collapsed relative to each other when in the tubular configuration such that the first region 1202 defines a second smaller diameter (not shown) along the length L. In other words, when the first region 1202 is in the reduced configuration, it is compressed transversely such that it is smaller and easier to insert and deliver (e.g., endoscopically) to the CBD 1250 or another targeted lumen.

The first region 1202 can be made from a material that enables the first region 1202 to be compressed elastically so that it can recover outwardly when the compressing force is removed and, thus, into contact with the wall of the targeted lumen. A balloon can also be deployed to facilitate expansion of the first region 1202 from the reduced configuration to the tubular configuration if desired. Alternatively, the first region 1202 can be formed of a shape memory alloy (e.g., nickel titanium) that has temperature-dependent shape memory and is capable of superelasticity. As used herein, “superelasticity” means the material can exhibit strains that may appear plastic in nature, but in fact can be completely recovered. There, following delivery to the CBD 1250 or other targeted lumen, the heat of the subject's body can trigger the first region 1202 to deploy and transition between the reduced configuration to the expanded tubular configuration. While certain specific embodiments are described herein, it will however be appreciated that any self-expanding stent technology suitable to the present applications can be employed.

The first region 1202 can also comprise a “drug-eluting” stent configured to deliver local chemotherapeutic compounds or other pharmaceutical compositions in addition to maintaining flow through the CBD 1250. For example, the first region 1202 can comprise an absorbable stent and/or a metal coated stent that is loaded with one or more drugs for treating cancer and/or to improve the performance of the stent by controlled delivery of the drug(s). The drug(s) can be loaded, for example, on the inside or outer surface of the first region 1202. Various iterations of drug-eluting stents are generally known in the art and non-limiting examples are described in the following references, which are incorporated by reference herein in their entireties: Lee, Drug-eluting stent in malignant biliary obstruction, J Hepato-Biliary-Pancreatic Surg 2009, 16(5): 628-632; Chung et al., Safety evaluation of self-expanding metallic biliary stents eluting gemcitabine in a porcine model, J Gastroenterology & Hepatology 2012, 27(2): 261-267; Mezawa et al., A study of carboplatin-coated tube for the unresectable cholangiocarcinoma, Hepatology 2000, 32(5): 916-923; and Tokar et al., Drug-eluting/biodegradable stents, Gastrointestinal Endoscopy 2011, 74(5): 954-958. In at least one exemplary embodiment, at least the first region 1202 of the stent 1200 is manufactured via 3-dimensional printing techniques using a drug-eluting material that is also negatively charged and hydrophilic to avoid the accumulation of bacteria therein.

Drug-eluting stents can be effective at not only maintaining flow through a lumen, but also preventing growth (or reducing the size) of a cancerous mass. Indeed, increased life spans have been recorded in subjects, as well as decreased tumor size, particularly when the one or more of the drugs comprises gemcitabine, which can be a general standard regime for advanced pancreatic and biliary cancers.

Additionally, as referenced above, where the first region 1202 comprises a drug-eluting stent, the first region 1202 can be optionally biodegradable or absorbable, or coated with an absorbable material such that the biodegradable or absorbable material is absorbed in vivo over a time period such as, for example, 3-6 months. In at least one embodiment, such absorbable material can comprise a PEGylated copolymer such as a poly(lactic acid)-poly(ethylene glycol)-poly(lactic acid) (PLA-PEG-PLA) copolymer. Optionally, the first region 1202 and/or the second region 1204 can be biodegradable or absorbable as described herein or otherwise known, but not drug-eluting.

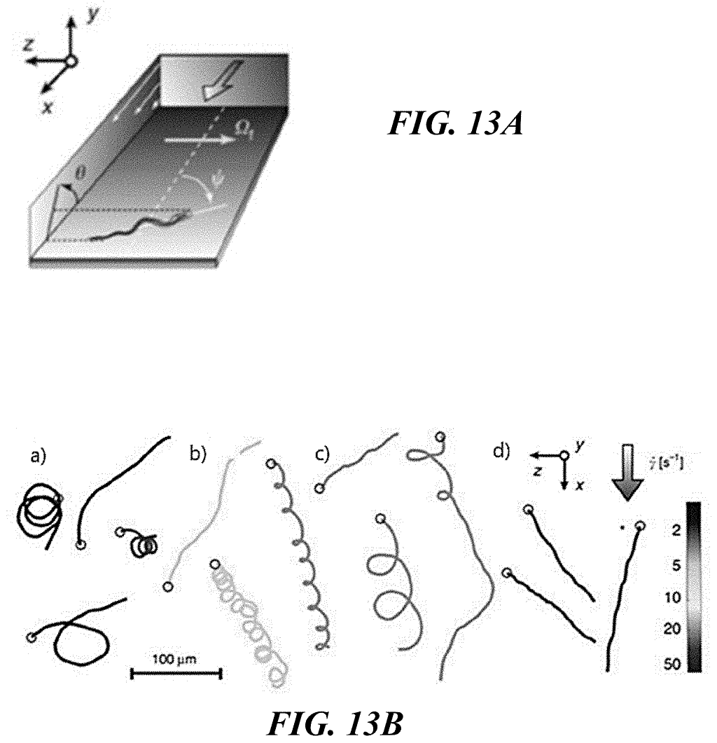

The interior surface 1302 of the first region 1202 can be configured to achieve one or more design goals (e.g., to control the direction of flow therethrough or otherwise manipulate the fluid dynamics therein). Controlling the direction of flow within the lumen 1203 of the first region 1202 and/or blocking bacterial in-flow are important to prevent contamination of the biliary and pancreatic systems, especially when treating cancer. Many different types of bacteria exist in the duodenum 1252, all of which can contaminate the biliary and pancreatic systems due to the unique ability of bacteria to swim upstream along a surface (see FIG. 13A). This ability to swim upstream along the walls of a structure is known as rheotaxis, which involves the reorientation of bacteria with respect to the flow gradient of a liquid. Rheotaxis is generally attributable to the asymmetry of the bacteria's shape.

Surfaces or sharp corners or edges are crucial for bacterial transport as they accumulate in such regions. With Escherichia coli (which is present in the human gastrointestinal system), four distinct swimming regimes have been identified that are distinguished via a critical shear rate (the shear exerted on a surface to which bacteria are swimming over time by an incident fluid). When swimming in a quiescent liquid (Regime 1, subpart a) of FIG. 13B), bacteria (such as E. coli) tend to swim in circles. As the shear rate increases (the shear applied to the surface by a flowing liquid), bacteria reorient themselves via rheotaxis and begin to change swimming trajectories. In Regime 2 shown in subpart b) of FIG. 13B, bacteria tend to swim upstream in a cycloidal motion. The third regime is characterized by oscillatory motion that is biased towards the positive vorticity direction (see subpart c) of FIG. 13B). Regime 4 exhibits a coexistence of oscillatory motion that can favor either the positive or negative vorticity direction, and an ability to switch between these two states (subpart d) of FIG. 13B).

With bacterial transport capabilities in mind, the interior surface 1302 of the first region 1202 can be configured to minimize or prevent bacterial transport upstream. In at least one embodiment, the interior surface 1302 of the first region 1202 can comprise a right-handed surface pattern that spirals inside of the lumen 1203 (e.g., similar to the rifling inside of a gun barrel, which causes the projectile to rotate). In the case of fluid flowing through the lumen 1203, a rifling along the interior surface 1302 of the first region 1202 would constantly disrupt the direction of flow, simultaneously disrupting the swimming dynamics of any bacteria. In at least one embodiment, the helix angle of the rifling can be tuned to the behavior of particular bacteria at issue. Notably, however, care can be taken with this design to avoid any sharp edges or corners being produced by modifying the interior surface 1302 topography of the first region 1202.

In certain embodiments, the first region 1202 is designed such that the incident fluid (e.g., bile) exerts a particular shear strain along the first region's 1202 interior surface 1302. For example, the first region 1202 can be designed to feel shear rates that encourage bacterial oscillatory motion (which increases the likelihood that the bacteria will detach from the interior surface 1302 that they are swimming up and thus be subjected to the downstream flow of fluid through the lumen 1203). This can be achieved through material selection (i.e. the incorporation of PXCMs and/or ASMAs), dimension selection, overall design, or one or more of the aforementioned.

The interior surface 1302 can also be configured to have a constantly changing geometry along a direction parallel to flow through the lumen 1203. Differences in the geometry of the interior surface 1302 can significantly disrupt the change in bacterial orientation and flow dynamics therethrough. Along these lines, in at least one embodiment, the first region 1202 of the stent 1200 (e.g., an interior surface 1302) comprises a one-way valve. A one-way valve allows a substance (e.g., bile) to flow through it in only one direction. It can comprise two openings, one of which allows a substance to enter the valve (e.g., upstream end 1202a) and one allowing the substance to exit.

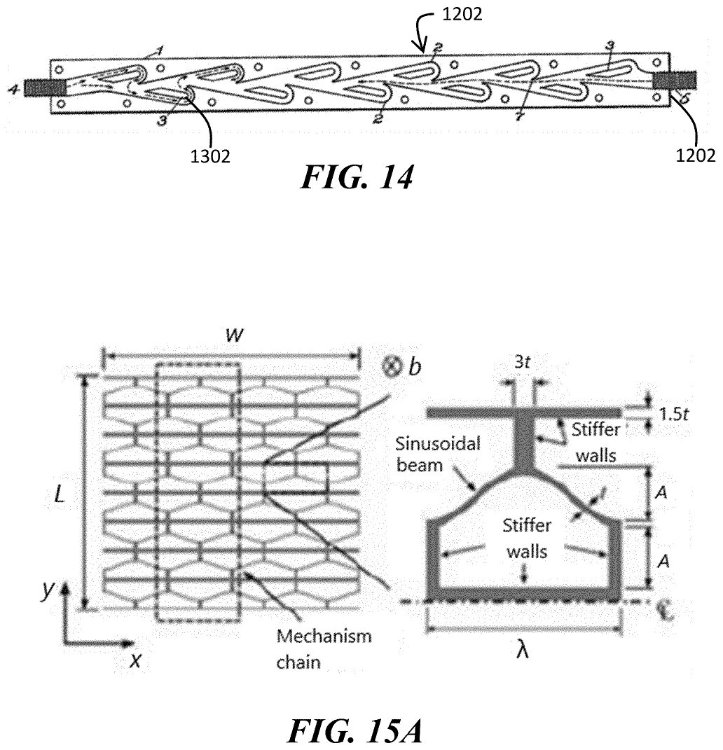

Many of the designs for one-way valves include moving parts such as the swing check valve (which uses the direction of fluid flow to effectively utilize a disc to open and close the valve), ball check valves (which use a ball to control the direction of fluid flow) and stop-check valves (which utilize changes in pressure to open and close the valve off to fluid flow). However, there are also one-way valves with no moving parts (“NMP valves”), such as the Tesla valve, which controls fluid flow direction with geometry via fluidics rather than with mechanical mechanisms. FIG. 14 shows at least one embodiment of a first region 1202 comprising a one-valve comprising a Tesla valve. Another example of a NMP valve is a Vortex-Diode valve.

In NMP valves, the mechanism that inhibits reverse flow has to do with the Reynolds number for turbulent flow. The Reynolds number of a flow is the ratio of the inertial forces to viscous forces within a fluid with a non-constant velocity gradient. The ability for a liquid to flow through an NMP valve is quantified by the valves diodicity and is given via the following ratio in Eq. (5):

$${D}_{i} = ( \frac{Δ {P}_{reverse}}{Δ {P}_{forward}} ) \qquad ( 5 )$$

where Δ_{Preverse} is the pressure loss in the reverse direction (e.g., the reverse direction of the Tesla valve shown in FIG. 14 is depicted as flow traveling from left to right) and ΔP_{forward} is the pressure loss in the forward direction (the forward direction for the Tesla valve in FIG. 14 is depicted as flow traveling from right to left). Accordingly, the pressure loss that occurs within NMP valves is a consequence of the inertial and viscous forces that occur in the flowing liquid.

In a Tesla valve, the concepts of converging and diverging flow are utilized to control the direction of fluid flow. The behavior of fluid flowing through a one-way valve such as this can vary depending upon whether the fluid is Newtonian or Non-Newtonian. When a fluid enters through opening 4 (see FIG. 14), it immediately diverges, which produces (by Bernoulli's principle) an increase in pressure along the direction of fluid flow until it reaches an obstruction (e.g., position 3), at which point the direction of fluid flow is reversed, causing it to crash into itself. When a fluid enters through opening 5, it is allowed to converge on itself, which decreases pressure along the direction of fluid flow and increases the velocity of the flow.

In at least one embodiment, the first region 1202 comprises a one-way valve comprising an interior surface 1302 defining the lumen 1203 and extending between the upstream end 1202a and the downstream end 1202b. The interior surface 1302 can comprise one or more interior walls of a fixed-geometry passive check valve configuration that permits free passage of fluid through the lumen 1203 in a first direction (in FIG. 13, right to left), but deters or prevents back flow of the fluid in a direction opposite the first direction (in FIG. 14, left to right). In certain embodiments, the first region 1202 comprises one or more NMP valves such as, for example, a Tesla Valve or a Vortex-Diode valve.

However, the conventional one-way valve designs do not possess a method of self-expansion upon increase of internal pressure, nor do they have a way by which they can open and close periodically such as the SO. To address this, certain embodiments of the stents 1200 hereof comprise one-way valves (and/or other components) composed of a material capable of geometric changes in response to a mechanical or chemical stimulus.

### Architected Materials

Architected materials are a family of materials that can effectively bridge material behavior across a broad range of length scales, making them advantageous for applications that involve one than one part having different sizes and shapes. These materials can be designed to exhibit unique properties including, but not limited to, a negative Poisson's ratio, simultaneous high strength and toughness, and energy dissipation, which can be accomplished by combining geometrical designs at different length scales with disparate material combinations to form a single architecture or hybrid material having properties that differ from those of the individual materials. In certain embodiments, such materials can be designed as on or more unit cells that exploit periodicity or randomness.

In certain embodiments, the stent 1200 comprises a subset of architected materials known as phase transforming cellular materials (PXCMs). See, e.g., Restrepo et al., Phase transforming cellular materials, Extreme Mechanics Letters 2015, 4: 52-60 (the “Restrepo Reference”), the entirety of which is incorporated herein by reference. As studies considering microfluidics suggest that NMP valves at the scale of the bile duct (˜10-3 m) depend more upon fluid surface tension, pressure, and viscosity (rather than motor function) to prevent reflex of duodenal contents into the biliary tree, stents 1200 comprising PXCMs can provide significant benefit. In at least one embodiment of the stent 1200, the first region 1202, the second region 1204, or both can comprise one or more types of PXCMs or unit cells thereof.

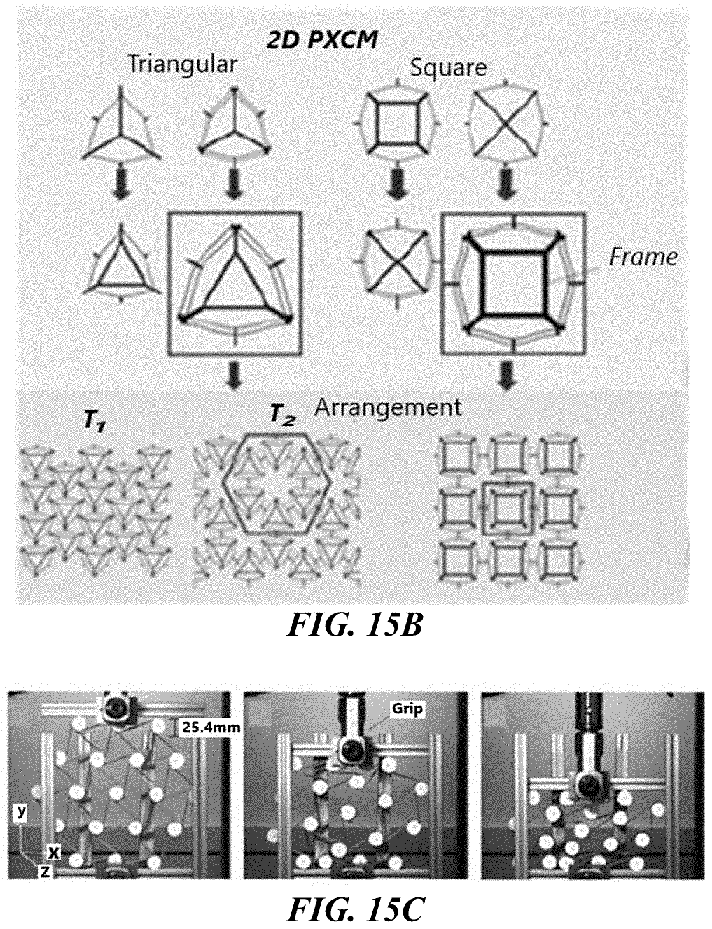

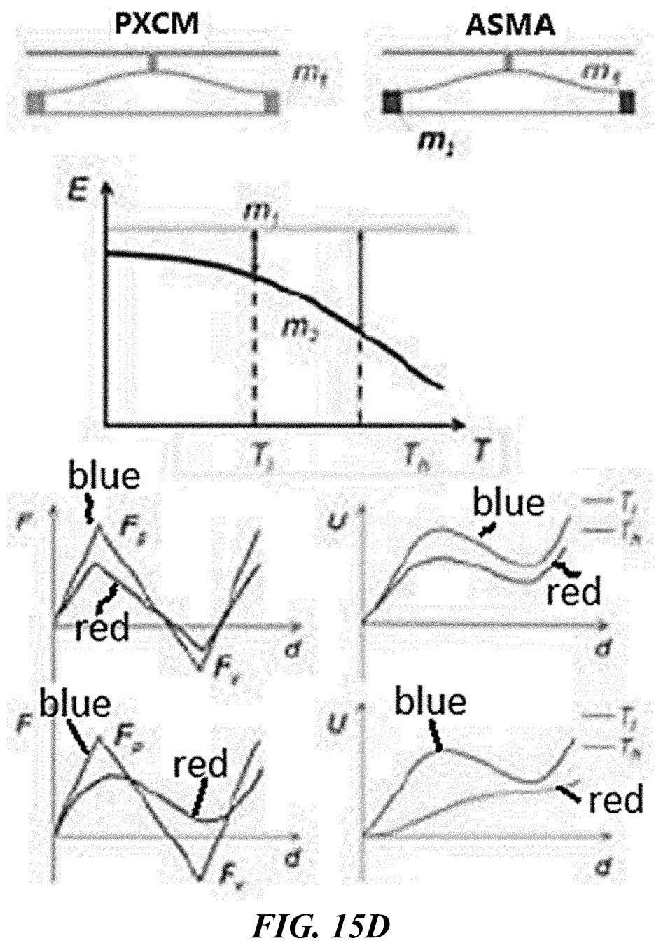

PXCMs have the potential for numerous energy dissipation and shape-morphing applications. For example, the unit cell described in the Restrepo Reference and shown in FIG. 15A, utilizes two bent beams of a sinusoidal shape which are connected to each other via a series of stiffening walls. These cells were assembled in series chains and are capable of dissipating energy quasistatically while remaining in the elastic regime (note that the SO opening and closing operates elastically).

Energy dissipation associated with the PXCM described in the Restrepo Reference was associated with first-order phase transformations that corresponded with sudden changes in the geometry of the PXCM unit cells during loading from one stable or metastable phase to another. These sudden changes are characterized by a sudden drop in load for a very small applied displacement (>>1 mm) which is known as snap-through. Each of these configurations for each unit cell is considered as a phase at the unit cell level, and transitions between these phases are considered to be phase transformations. It is important to note that, in the PXCM material described in the Restrepo Reference, the sinusoidal beams functioned as the snapping mechanism, which affords it the unique capabilities of recoverable phase transformation and energy dissipation that are unavailable with monolithic materials.

Energy dissipation in functionally 2-dimensional PXCMs was also investigated in Zhang et al., Energy dissipation in functionally two-dimensional phase transforming cellular materials, Scientific Reports 2019, 9(1): 1-11 (the “Zhang Reference”), and Hector et al., Mechanics of chiral honeycomb architectures with phase transformations, J of Applied Mechanics 2019, 86(11) (the “Hector Reference”), both of which are incorporated herein by reference in their entireties. In the Zhang Reference and as shown in FIG. 15B, a sinusoidal beam in two separate designs for a 2-dimensional PXCM was utilized that had the following unit cell geometries: The S-type with four axes of reflectional symmetry (based on a square motif), and the T-type with six axes of reflectional symmetry (based upon a triangular motif). Finite element results indicated that 2-dimensional PXCMs consisting of these unit cells were also capable of energy dissipation for loads applied along their axes of symmetry while remaining within the elastic limit. The Hector Reference discloses a 2-dimensional PXCM having a tape spring ligament arranged into a chiral topology (see FIG. 15C). Notably, a tape spring ligament is a compliant structure with an initial transverse curvature and was shown to be capable of recoverable energy dissipation regardless of the number of cells in the material (because a single tape spring ligament on its own was capable of snap through).

Accordingly, any of the PXCMs described herein or otherwise known can be incorporated into the stent 1200 as desired. A single type of PXCM can be employed or, alternatively, the first and/or second regions 1202, 1204 can each comprise two or more different types of PXCMs (e.g., those disclosed in the Zhang Reference, the Restrepo Reference, the Hector Reference, and/or any other type of PXCM now known or hereinafter developed) to achieve the desired compliance, responsiveness, shape morphing ability, superelasticity, and other mechanical properties or configurations.

The resulting stent 1200 (or portion thereof that contains the PXCM) exhibits simultaneous high strength and toughness and the ability to dissipate energy for loads applied along its axis (e.g., where a tumor or other cancerous growth increases in pressure over time as it grows). Furthermore, due to PXCM's ability to shape morph, the stent 1200 (e.g., first region 1202) can also adapt to a particular shape of the tumorous tissue within the targeted bile duct (e.g., CBD 1250).

In at least one embodiment, the first and/or second regions 1202, 1204 is/are designed to impart a specific radial stiffness. Such radial stiffness can be important for resisting concentric or eccentric radial forces and maintaining the shape of the first region 1202, for example, once deployed. In at least one embodiment, the first region 1202 is configured to increase its stiffness as it is subjected to an increased load due to a growing cancerous or other mass (e.g., a circumferential load, a concentric radial force, or an eccentric radial force). FIG. 12 shows three embodiments of cross-section 1206 designs 1206a, 1206b, 120c of a first region 1202 comprising PXCM that can achieve such effect.

At least one benefit that can be achieved through the incorporation of PXCM into the stent 1200 is decreasing the incidence of jaundice in the subject. Among the numerous outcomes from bile duct cancer, one of particular concern is jaundice, which is a direct consequence of long term (about 3-6 months) obstructions in the CBD. The CBD typically operates at a low internal pressure (about 3-7 mmHg); however, if obstructed, pressures can reach up to 22 mmHg. When the pressure increases inside the CBD, its walls and those of the hepatic ducts in the liver become more permeable, which can enable flow of bile out of the biliary system and into the blood stream. Jaundice is caused by long term leakage of bile into the blood stream, resulting in blood infections, abnormalities in liver function, and yellowish coloration in the eyes and skin. Because cancer patients undergoing chemotherapy have a compromised immune system, this can be exceptionally problematic. In most cases, presentation of jaundice requires that chemotherapy treatment be put on hold in favor of managing any resultant infections with antibiotics, which results in the growth of the cancerous tumors and ultimately patient death.

In at least one exemplary embodiment where at least the first region 1202 comprises one or more types of PXCM, the PXCM can be configured in a manner to replicate the behavior of a biological one-way valve within the first region 1202; for example, be designed to be metastable and, thus, capable of transforming between the open and closed configurations without the need for a load. This, especially when taken in conjunction with pulsating behavior of the second region 1204, results in a stent 1200 of advantageous properties. The stent 1200 can prevent or significantly delay an unmitigated infection in the subject brought on by jaundice, for example, thus enabling cancer treatments such as chemotherapy to proceed and increasing the subject's overall chance of survival.

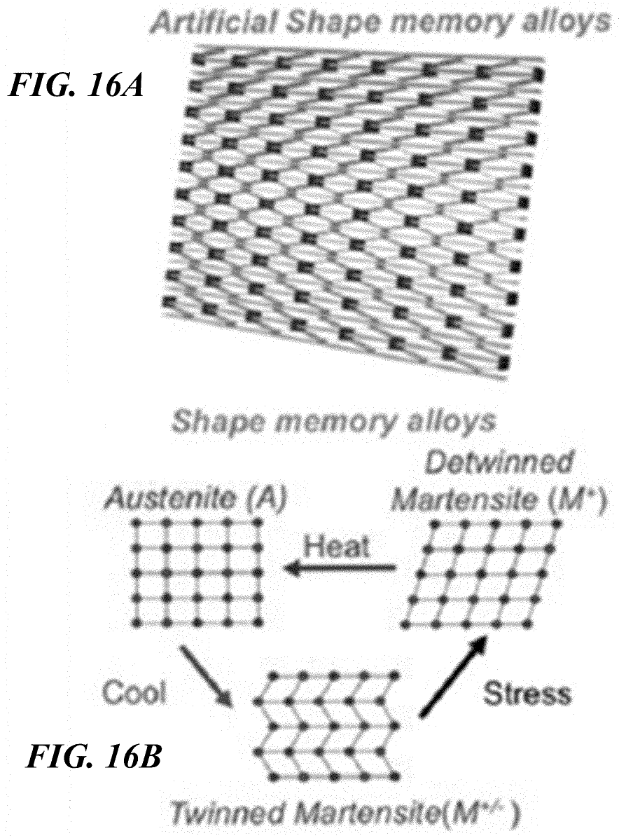

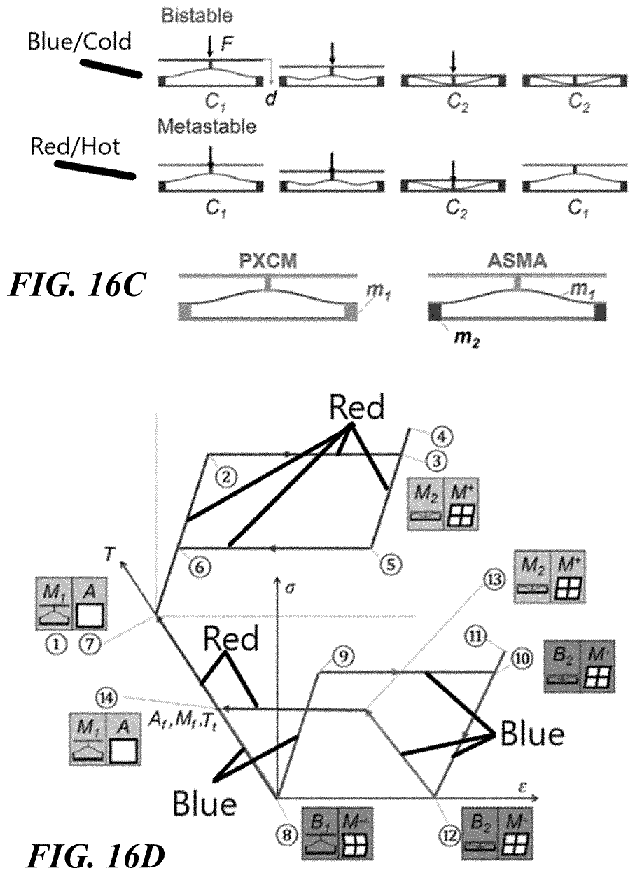

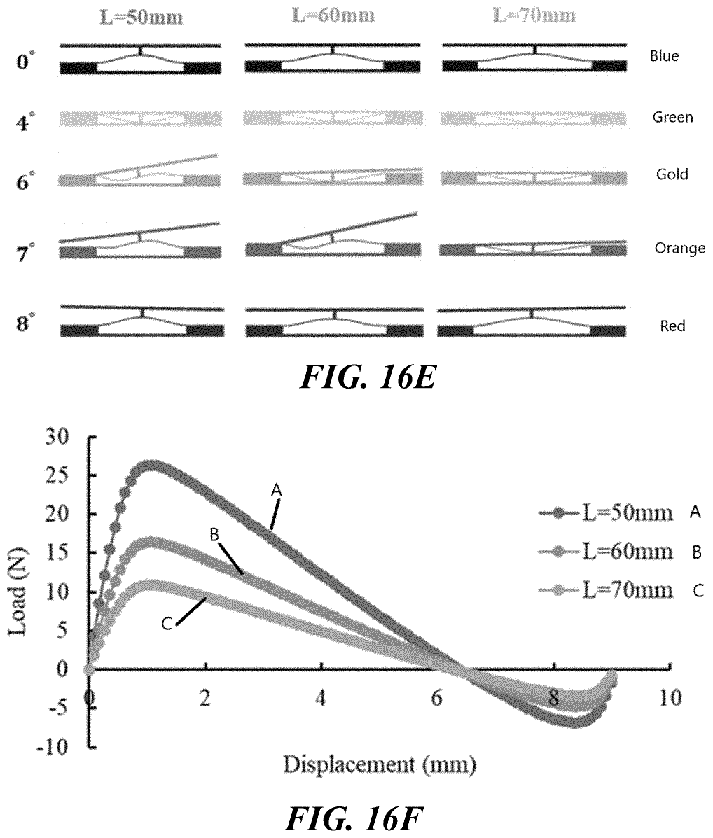

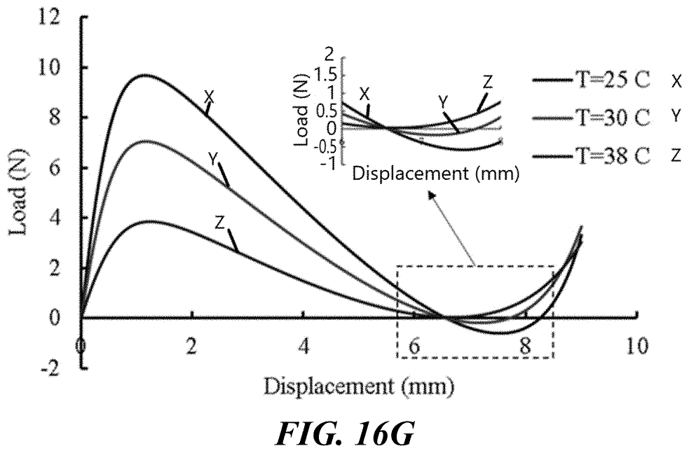

Additional materials that may be used to form all or part of the stent 1200 are architected material analog for shape memory alloys (SMAs or ASMAs), for example, those described in Zhang et al., Architected materials analogs for shape memory alloys, Matter 2021, 4(6): 1990-2012. (the “Zhang ASMA Reference”). ASMAs comprise a periodic cellular material that mimics the salient behaviors of shape memory alloys, such as superelasticity and shape memory. In certain embodiments, the architected material analog for ASMAs comprises two materials (see FIG. 16C) and is capable of exhibiting both of the salient behaviors of shape memory alloys (SMAs)—namely, superelasticity and the shape memory effect.

ASMA materials can achieve the shape memory function by undergoing a phase change of the alloy at a transition temperature while in the solid state (e.g., without melting).

For example, an ASMA material can comprise a block of sinusoidal beam that is anchored in supports made of a material whose storage modulus decreases at a faster rate with increasing temperature than that of the beam. At low temperatures, the storage moduli of the two constituent materials have comparable magnitudes and the block exhibits two stable configurations. The block can transition elastically from one stable configuration to the other via a snap-through in response to an external load. Above a critical temperature, the storage modulus of the supports is sufficiently low such that the second stable configuration becomes unstable, and the block returns to its first stable configuration without any external load. These responses of the block result in shape memory alloy-like material behavior in an ensemble of such blocks. It will be noted that such an ASMA material can comprise the two materials that were used to construct the PXCM described in the Restrepo Reference (see FIG. 16A).

Shape memory alloys rely on changes at the molecular level to exhibit temperature-dependent mechanical behavior. For example, a lower temperature phase can be referred to as martensite in which the position of the particles within the crystal structure of the solid can be rearranged by applied mechanical forces. Thus, in the lower temperature, martensite phase, the material can be malleable and can be bent and deformed at will. Consider a shape memory alloy at the molecular level, starting in its unstressed, twinned, martensitic phase shown in FIG. 16B (see also FIG. 16D, point 8). Once a stress is applied that is capable of inducing plastic deformation in the shape memory alloy, the shape memory alloy enters its detwinned, martensitic phase (see FIG. 16B and FIG. 16D, points 9, 10, and 12). When heat is introduced to the plastically deformed shape memory alloy (FIG. 16D, point 13), a phase transformation occurs at the molecular level which transitions the shape memory alloy to an Austenitic phase, which allows the shape memory alloy to recover elastically (again, FIG. 16B and FIG. 16D, point 14). Additionally, the shape developed in the austenite phase persists after the ASMA material is cooled and returns to the malleable and flexible martensite phase.

These characteristics can be employed to achieve heat-driven transitions between metastable and bistable mechanisms, Q, of an ASMA (FIGS. 16C-16G). The mechanical behavior exhibited by a bistable ASMA (blue, FIG. 16C) can be analogous to a shape memory alloy transitioning from a twinned martensitic phase to a detwinned martensitic phase. This corresponds to the blue stress-strain curve shown in FIG. 16D. However, when heat is applied to the bistable ASMA, since one of the consisting materials has a temperature dependent Young's Modulus, it can now behave as a metastable ASMA (red, FIG. 16C) which corresponds to the red stress-strain curve shown in FIG. 16D. The transfer between bistable ASMA and metastable ASMA with an increase in heat is analogous to the phase transformation of a shape memory alloy from a detwinned martensitic phase to an elastic martensitic phase. Further, the stiffness and peak load decrease with increasing wavelength (L) (see FIG. 16F), and that an ASMA unit cell can convert from a bistable mechanism to a metastable mechanism when the minimum load (generally the most negative load) ceases to be negative and becomes positive as shown in the subfigure in FIG. 16G, which corresponds to the change in the mechanical behavior of the L=50 mm ASMA cell as temperature increases.

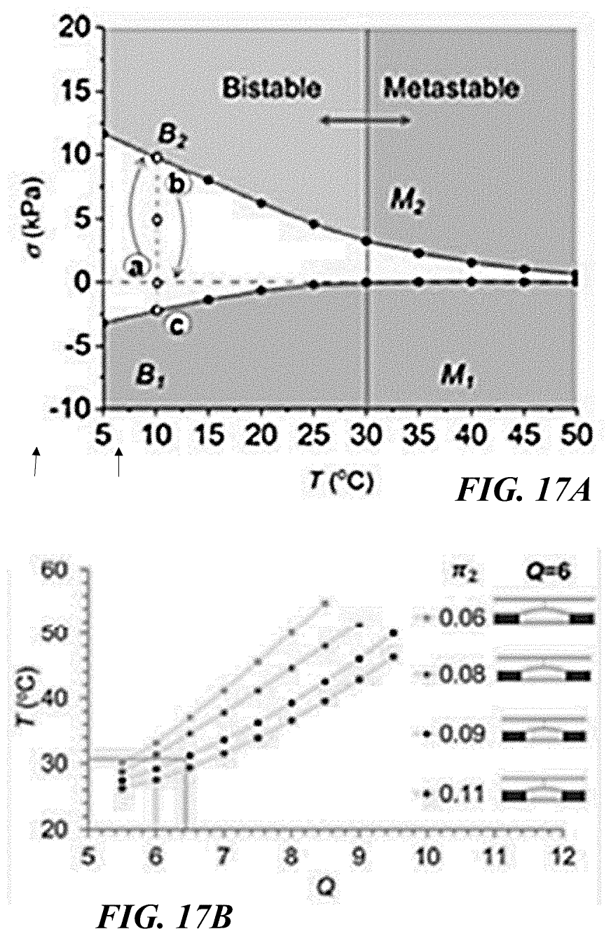

At least one benefit of employing ASMAs in the composition of the stent 1200 is that ASMAs can be designed to exhibit a specific mechanism, Q, for a particular temperature and applied stress (see, e.g., FIGS. 17A and 17B). The peak stress of an ASMA as a function of the temperature is shown in FIG. 17A. Here it can be seen that there is a temperature-dependent boundary between the bistable and metastable mechanisms, Q, of the ASMA. FIG. 17B provides a design map of the ASMA featuring the mechanism, Q, as a function of the temperature. Here it is seen that the mechanism Q of an ASMA increases (tends towards a bistable mechanism) as the temperature increases, and vice versa for decreasing temperature.

Accordingly, use of one or more PXCMs (e.g., one or more ASMAs) in the composition of the first and/or second regions 1202, 1204 can allow for the customization of mechanical properties thereof such that the stent 1200 can be tuned to a specific subject and/or application. For example, where the first region 1202 comprises ASMAs, its configuration can be tuned to provide a specific radial stiffness. Additionally, there, the first region 1202 can be additionally designed to increase its stiffness as it is subjected to an increased load due to a growing cancerous or other mass (e.g., a circumferential load, a concentric radial force, or an eccentric radial force).

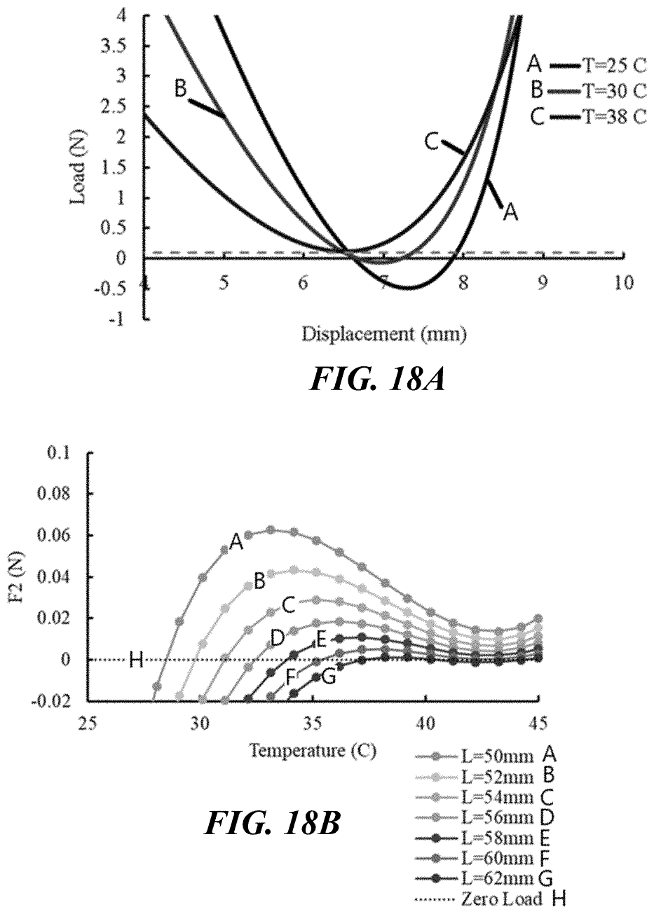

When the stent 1200 comprises one or more ASMAs, such ASMAs can additionally be designed to exert work on an external resistance and/or compressive force (e.g., to facilitate the stent clearing clogged debris via a swallowing motion). In such embodiments, when an external resistance is not exerted on the ASMA, it can transition from a bistable to a metastable mechanism when the temperature is sufficiently increased so the minimum load (F2) of the cells is no longer negative (see FIGS. 16G and 18A-18B). However, when an external resistance and/or compressive force is applied to such an ASMA cell, the minimum load of the cell must overcome that resistance to return to its original configuration (see FIG. 18A), noting that for any ASMA design, there will be an upper bound on an achievable F2 value and, thus, an upper bound on the external resistance (force) that the ASMA cell can do work against.

This is illustrated in FIG. 18B, which depicts the values of F2 for several ASMA designs at several different temperatures. In the case of each ASMA design, there is a maximum value on F2 that is achievable. Additionally, if the wavelength (L) of the ASMA is decreased, the maximum value of F2 that can be achieved by that ASMA cell increases.

In at least one embodiment, the first region 1202 of the stent 1200 comprises one or more ASMA unit cells designed to exhibit a reversal in displacement in response to an increase in temperature. The ASMA unit cells can all have the same or different wavelength (e.g., 35 mm, 40 mm, 45 mm, 50 mm, 55 mm, and/or 60 mm). In certain embodiments, the stent 1200 only comprises the first region 1202 comprising one or more ASMA unit cells. In other embodiments, the stent 1200 comprises the first region 1202 comprising one or more ASMA unit cells and the second region 1204.

Such ASMA unit cells can, for example, line at least a portion of an interior surface of the first region 1202 that defines the lumen 1203. The ASMA unit cells can be arranged in series or in any other pattern on the interior surface of the first region 1202. In at least one embodiment, each ASMA unit cell positioned on the interior surface of the first region 1202 is within 10 mm of another ASMA unit cell. In certain embodiments, the ASMA unit cells can be positioned on the interior surface of the first region 1202 in such a manner that a staggered reversal in displacement is achieved in response to increasing temperature within the lumen. In this manner, when an obstruction is present within the lumen 1203 of the first region 1202, the obstruction applies external resistance and/or compressive force to the ASMA unit cells, and such unit cells respond with motion that pushes against the obstruction (i.e. the resistive and/or compressive force). Where the ASMA unit cells are positioned in a series or other pattern on the interior surface of the lumen 1203, the pressure against the obstruction by each of the ASMA unit cells can work in concert to push along and out of the lumen 1203, mimicking peristaltic behavior and effectively clearing the stent 1200. As these ASMA unit cells can be designed to be temperature responsive, this can also be achieved as a temperature-controlled response.

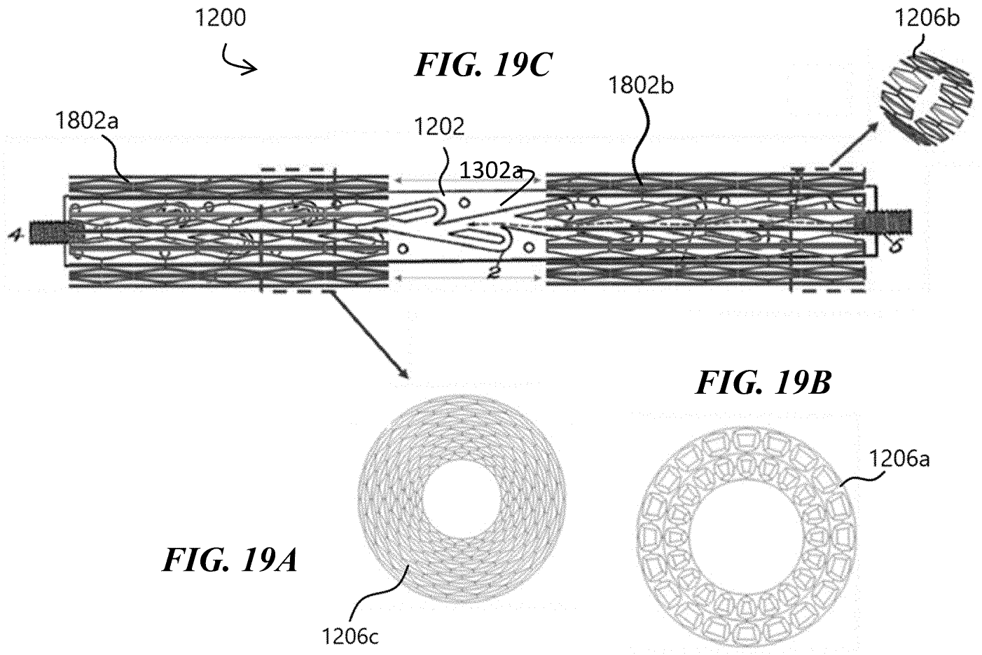

Now referring to FIGS. 19A-C, the stent 1200 can additionally comprise one or more PXCM coverings 1802 positioned around a circumference of the first region 1202. Where more than one PXCM coverings 1802 are employed (e.g., PXCM 1802a, 1802b), such coverings 1802 can be positioned in different circumferential locations around the first region 1202 as shown in FIGS. 19A-C, or one or more may be positioned concentrically such that they overlap each other to some extent.

The PXCM covering 1802 can be used to “lock” and “unlock” regions of convergent flow in an NMP valve. Note, for example, that the embodiment of stent 1200 in FIG. 19A comprises an interior surface 1302a comprising a Tesla valve configuration (this NMP configuration is merely shown by way of example to illustrate the concept and is not intended to be limiting). In operation, the first region 1202 comprising the Tesla valve 1302a promotes bile flow from the biliary tree to the duodenum 1252 and deters back flow from the duodenum 1252 (i.e. back into the outlet 1204b of the second region 1204 and into the lumen 1203 of the first region 1202). In at least one embodiment, the PXCM covering 1802 interacts with the changing local levels of CCK to squeeze and relax, thereby simultaneously preventing and allowing bile flow through the lumen 1203 at points of converging flow.

Methods for treating a subject having a wholly or partially compressed or obstructed duct are also provided. The subject can be, for example, experiencing pancreatic cancer, CC, or another type of cancer. The wholly or partially compressed or obstructed duct can be a result of such cancer, for example, a cancerous growth or tumor within, on, or near the targeted duct.

In at least one embodiment, such a method comprises inserting (or having inserted) any of the variations of the stents 1200 described herein into a targeted duct of the subject. Where the stent 1200 is a self-expanding stent, the stent 1200 can be inserted in its reduced configuration and the method can further comprise the step of expanding, or allowing to expand, the self-expanding stent in the targeted duct.

Where the subject is experiencing, or at risk of experiencing, bile duct cancer, the targeted duct can be a CBD, and the method further comprises positioning the second region 1204 of the stent 1200 within an ampulla of Vater of the subject. The method can further comprise administering to the subject a treatment for the cancer (e.g., chemotherapy, chemoradiotherapy, or the like).

Methods for treating cancer in a subject are also provided. In certain embodiments, the cancer is pancreatic cancer, CC, or another type of cancer. In certain embodiments, a method for treating cancer in a subject comprises inserting (or having inserted) any of the variations of the stents 1200 described herein into a targeted duct of the subject. Where the stent 1200 is a self-expanding stent, the stent 1200 can be inserted in its reduced configuration and the method can further comprise the step of expanding, or allowing to expand, the self-expanding stent in the targeted duct.

Methods for treating jaundice in a subject are also provided. Such a method can comprise inserting (or having inserted) any of the variations of the stents 1200 described herein into a targeted duct of the subject. Where the stent 1200 comprises one or more ASMAs within the first region, the stent 1200 can replicate and replace lost peristaltic behavior within that area and, thus, assist in keeping the lumen and related ducts (e.g., a bile duct) free of debris and/or obstruction.

Methods for clearing an obstruction from a stent positioned within a subject are also provided. In at least one embodiment, the stent comprises any of the stents described herein where the first region comprises one or more ASMA unit cells designed to exhibit a reversal in displacement in response to an increase in temperature. For example, the one or more ASMA unit cells can line at least a portion of an interior surface of the first region that defines the lumen of the stent. The method can comprise applying external/compressive force to a first set of ASMA unit cells (e.g., via an obstruction within the stent); pushing against the external force (e.g., the obstruction) with the first set of ASMA unit cells to move the external force in a direction through the lumen; applying the external force to a second set of ASMA unit cells (e.g., such second set of ASMA unit cells being positioned at a location further along the lumen of the stent than the first set of ASMA unit cells); and pushing against the external force (e.g., the obstruction) with the second set of ASMA unit cells to move the external force in the direction through the lumen. These steps can be repeated until the external force/obstruction is expelled from the lumen of the stent. In certain embodiments, the method further comprises applying heat to the subject at a location adjacent to the stent to activate the (e.g., first and second sets of) ASMA unit cells.

All patents, patent application publications, journal articles, textbooks, and other publications mentioned in the specification are indicative of the level of skill of those in the art to which the disclosure pertains. All such publications are incorporated herein by reference to the same extent as if each individual publication were specifically and individually indicated to be incorporated by reference.

While certain embodiments of the present disclosure have been shown and described herein, it will be apparent to those skilled in the art that such embodiments are provided by way of example only. It is not intended that the claimed invention be limited by the specific examples provided within the specification.

While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein, which depend upon a variety of conditions and variables. It should be understood that various alternatives to the embodiments of the invention described herein may be employed in practicing the invention. It is, therefore, contemplated that the invention shall also cover any such alternatives, modifications, variations or equivalents. It is intended that the following claims define the scope of the invention and that methods and structures within the scope of these claims and their equivalents be covered thereby.

The use of headings and subheadings is solely for ease of reference and is not intended to limit the scope of the disclosure under a given heading or subheading to the subject matter set forth there under. Rather, disclosure under any heading or subheading is applicable to all subject matter herein, unless expressly indicated otherwise or contradicted by context.

### Certain Definitions

Unless defined otherwise, all technical and scientific terms used herein have the same meaning as commonly understood by one of skill in the chemical and biological arts.

As used herein and in the appended claims, the singular forms “a,” “and,” and “the” include plural referents unless the context clearly dictates otherwise. Thus, for example, reference to “a compound” includes a plurality of such compounds. When ranges are used herein for physical properties, such as molecular weight, or chemical properties, such as chemical formulae, all combinations and sub-combinations of ranges and specific embodiments therein are intended to be included.

The term “about,” when referring to a number or a numerical range, means that the number or numerical range referred to is an approximation within experimental variability (or within statistical experimental error), and thus the number or numerical range may vary between 1% and 15% of the stated number or numerical range.

The term “comprising” (and related terms such as “comprise” or “comprises” or “having” or “including”) is not intended to exclude in certain embodiments an embodiment of any compound, composition, method, process, or the like that may “consist of” or “consist essentially of” the described features.

A “subject” or “patient,” as used herein, is a mammal, preferably a human, but can also be an animal.

The terms “treat,” “treating,” or “treatment” include reducing, alleviating, abating, ameliorating, relieving, or lessening the symptoms associated with cancer in either a chronic or acute therapeutic scenario.

### EXAMPLES

The following examples serve to illustrate the present disclosure and are not intended to limit its scope in any way.

### Example 1

### ASMA Unit Cell Design

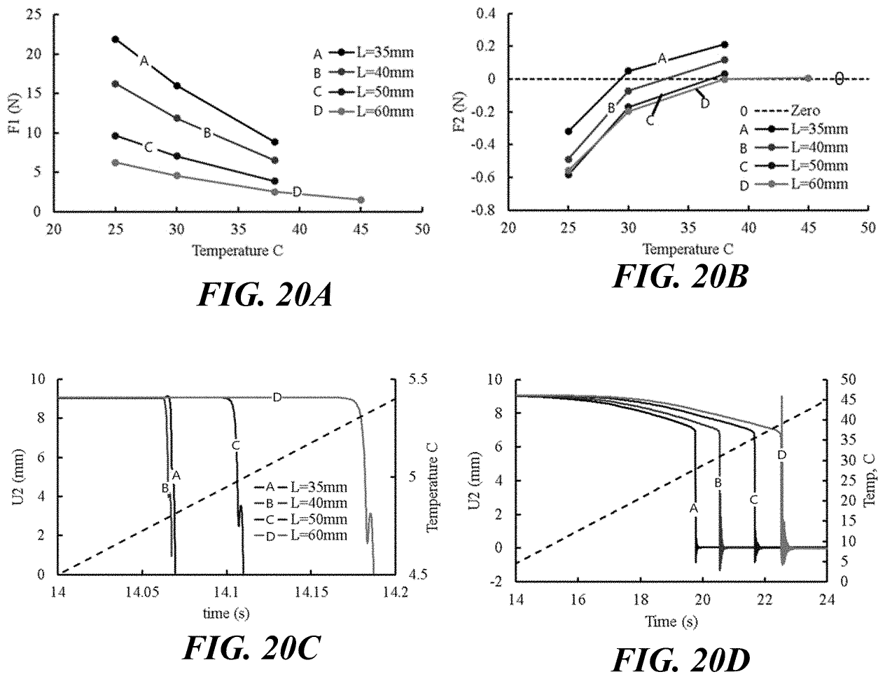

Four ASMA unit cells were designed, the mechanical behavior of which is outlined in FIGS. 20A-20D. All loads and displacements on each ASMA was calculated via the finite element analysis with an ABAQUS/CAE three-dimensional finite element model software package.

The maximum transition load (F1) of the ASMA designs as a function of temperature is shown in FIG. 20A. Note that curves A, B, C, and D correspond to an L=35 mm, L=40 mm, L=50 mm, and L=60 mm, respectively. FIG. 20A supports that, for these designs, there is an inverse relationship between temperature and the load required to collapse each cell (F1).

FIG. 20B depicts the minimum load (F2) of the ASMA designs as a function of the temperature, and supports that each ASMA design works without an external resistance as the F2 value of each design becomes a positive load at a temperature of 45° C.

Two types of boundary conditions were used in selecting the ASMA designs, the first of which enabled each ASMA design to rotate. The displacement behavior of each ASMA cell from this group is shown in FIG. 20C. The second type of boundary condition did not enable rotations in the ASMA cells as they returned to their original shape. The displacement behavior of each ASMA cell associated with the second type of boundary condition (no rotation) is shown in FIG. 20D. In this case, the temperatures at which each cell converted from a bistable cell to a metastable cell was much more discrete than those shown in FIG. 20C (rotation). However, regardless of the choice of boundary conditions, by changing the wavelength (L) of the ASMA cells, the ASMA cells can be designed to exhibit a staggering reversal in displacement when subjected to increasing temperature. Such a phenomenon can be used to push an object along a tube lined with such ASMAs.

### Example 2

### Application of External Resistance

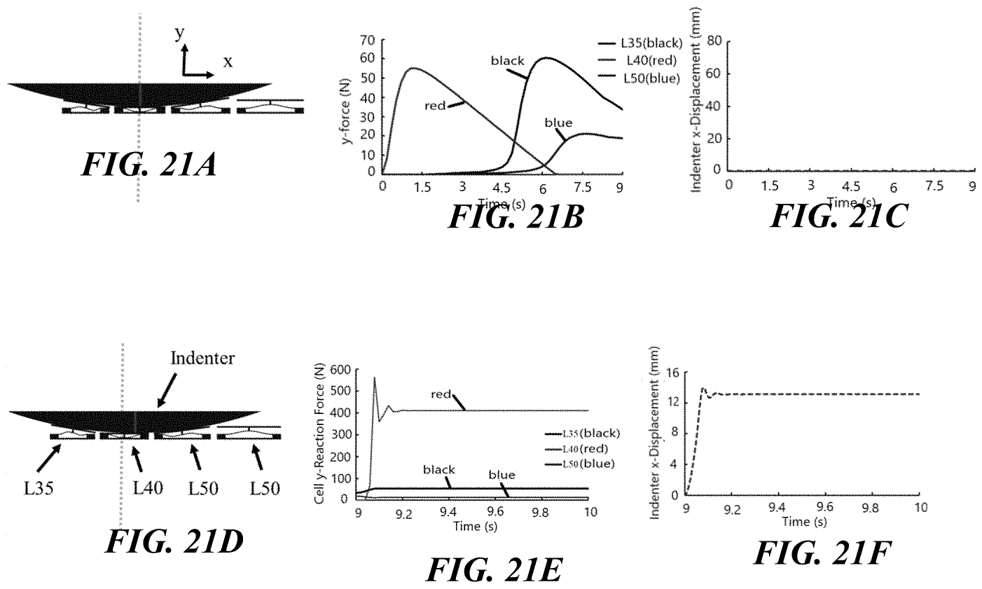

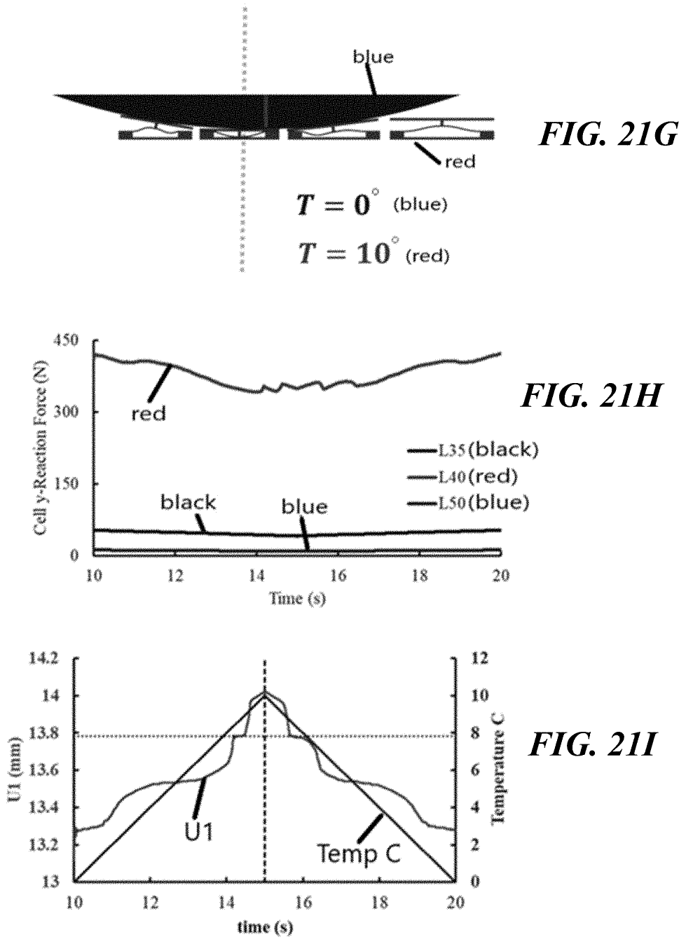

An indenter with a radius of 500 mm (10 times that of the L=50 mm ASMA cell) was used as external resistance acting on the ASMA cells. The ASMA cells were each separated by 10 mm in a straight row and an axisymmetric boundary condition was assumed (depicted in FIG. 21A). The indenter and the ASMA cells were modeled with 2D plane strain shell elements. These finite element simulations were broken into 3 stages: (1) an initial indentation stage (stage 1 shown in FIG. 21A); (2) an equilibrium stage (stage 2 shown in FIG. 21D); and (3) an activation stage (stage 3 shown in FIG. 21G).

In stage 1/initial indentation stage, the acrylic indenter was loaded into the row of ASMA cells strictly in the y direction (to emulate an obstruction becoming clogged in an ASMA-lined stent hereof), and the stage was run with dynamic implicit. FIG. 21B shows the forces observed in the y-direction, which illustrates how the different ASMA cells competed with each other. Note that at 9 seconds, L35 has a higher load than either L40 or L50, which supports that L35 was pushing back the hardest on the acrylic indenter at that point. FIG. 21C supports there was no displacement in the x direction for the indenter during this stage 1.

Immediately following stage 1, stage 2 was run using a dynamic explicit solver in which the indenter was allowed to move in the x-direction in its indented state (see FIG. 21D). The indenter moved in the positive x direction until and equilibrium position was identified. FIG. 21E shows the y-reaction forces that each cell exerted on the acrylic indenter. FIG. 21F supports that the indenter found an equilibrium position as the x-indenter displacement plateaued around 13.1 mm.

Stage 3 involved activating the ASMA cells with temperature changes. In the case of FIGS. 21H and 21I, the temperature was increased by 10° C., which did increase the x-displacement exhibited by the indenter, but not sufficient to permanently move the indenter into a new global equilibrium point.

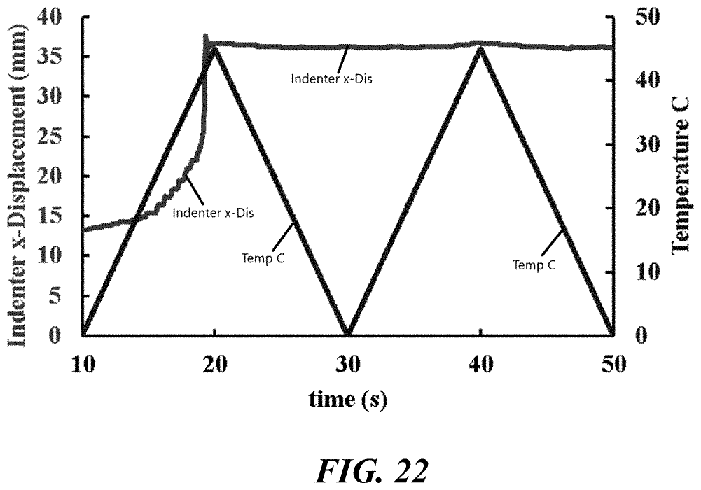

To observe additional displacement in the acrylic indenter, the temperature was increased to the limit on the base materials of the ASMAs (45° C.). FIG. 22 displays the results of this study with respect to stage 3. There were two 45° C. temperatures cycles in which the temperature was brought to 45° C. and back down to 0° C. twice. Considerable displacement was achieved by the indenter and maintained through the second cycle; however, this displacement was not repetitive due to the forces required to move the indenter further out of its current equilibrium point.

1. A stent comprising:
a first region comprising an upstream end, a downstream end, and a lumen extending a length between the upstream end and the downstream end, the first region having an elongated tubular configuration where each of the downstream end and the upstream end are expanded radially and the first region defines a first diameter along the length of the lumen; and
a second region coupled with the downstream end of the first region, defining an outlet that is in fluid communication with the lumen of the first region, wherein the second region is comprised of one or more phase transforming cellular materials (PXCM) configured to move the outlet between an open configuration and a closed configuration in response to a change in one or more of an energy imbalance in the PXCM, a change in pressure through an interior of the second region, and a change in a local concentration of cholecystokinin (CCK).

2. The stent of claim 1, wherein moving between an open configuration and a closed configuration emulates the mechanics and associated geometric changes of an ampulla of Vater during contraction and relaxation of a Sphincter of Oddi (SO).

3. The stent of claim 1, wherein the first region further comprises a reduced configuration where each of the downstream end and the upstream end are collapsed relative to each other in the tubular configuration and the first region defines a second diameter along the length of the lumen, wherein the second diameter is less than the first diameter of the elongated tubular configuration.

4. The stent of claim 3, wherein the first region is configured for self-expansion from the reduced configuration to the tubular configuration.

5. The stent of claim 1, wherein the first region is configured to increase a stiffness when subjected to a circumferential load, a concentric radial force, or an eccentric radial force.

6. The stent of claim 1, wherein the first region comprises one or more PXCM or architected material analog for shape memory alloy (ASMA) unit cells.

7. The stent of claim 1, wherein the first region further comprises a one-way valve.

8. The stent of claim 7, wherein the one-way valve comprises an interior surface defining the lumen and extending between the upstream end and the downstream end, the interior surface comprising one or more interior walls of a fixed-geometry passive check valve configuration to permit free passage of fluid through the lumen in a first direction but deter or prevent back flow of the fluid in a direction opposite the first direction.

9. The stent of claim 7, wherein the first region further comprises at least one PXCM covering positioned around a circumference of the first region, each of the PXCM coverings configured to compress or decompress the underlying first region in response to a change in local concentration of CCK to restrict or allow, respectively, fluid flow through the first region.

10. The stent of claim 1, wherein the first region and the second region are biodegradable.

11. The stent of claim 1, wherein the first region comprises a drug eluting stent.

12. A stent comprising:
a first region comprising:
an upstream end, a downstream end, a lumen extending a length between the upstream end and the downstream end, and an interior surface extending between the upstream end and the downstream end and defining at least a portion of the lumen,
wherein the interior surface comprises one or more interior walls of a fixed geometry passive check valve configured to permit free passage of fluid through the lumen in a downstream direction but deter or prevent back flow of the fluid in an upstream direction, and the first region is movable between a tubular configuration having a first diameter and a reduced configuration having a second diameter, wherein the tubular configuration of each of the downstream end and the upstream end are expanded radially, in the reduced configuration each of the downstream end and the upstream end are collapsed relative to each other in the tubular configuration, and the second diameter is less than the first diameter;
a second region coupled with the downstream end of the first region, defining an outlet in fluid communication with the lumen of the first region, wherein the second region is comprised of one or more phase transforming cellular materials (PXCM) configured to move the outlet between an open configuration and a closed configuration in response to a change in one or more of an energy imbalance in the PXCM, a change in pressure through an interior of the second region, and a change in a local concentration of cholecystokinin (CCK); and
at least one PXCM covering positioned around a circumference of the first region and configured to compress or decompress the underlying first region in response to a change in concentration of CCK to restrict or allow, respectively, fluid flow through the first region.

13. A method for treating a subject having a wholly or partially compressed or obstructed duct comprising:
providing a self-expanding stent comprising:
a first region comprising an upstream end, a downstream end, and a lumen extending a length between the upstream end and the downstream end, wherein the first region is movable between a tubular configuration having a first diameter and a reduced configuration having a second diameter, where in the tubular configuration each of the downstream end and the upstream end are expanded radially, in the reduced configuration each of the downstream end and the upstream end are collapsed relative to each other in the tubular configuration, and the second diameter is less than the first diameter, and
a second region coupled with the downstream end of the first region, defining an outlet in fluid communication with the lumen of the first region, wherein the second region is comprised of one or more phase transforming cellular materials (PXCM) configured to move the outlet between an open configuration and a closed configuration in response to a change in one or more of an energy imbalance in the PXCM, a change in pressure through an interior of the second region, and a change in a local concentration of cholecystokinin (CCK);
inserting, or having inserted, the self-expanding stent in a reduced configuration into a targeted duct of the subject; and
expanding, or allowing to expand, the self-expanding stent in the targeted duct.

14. The method of claim 13, wherein the targeted duct is a common bile duct and the method further comprises positioning the second region of the self-expanding stent within an ampulla of Vater of the subject.

15. The method of claim 13, wherein the outlet of the second region of the stent moving between an open configuration and a closed configuration emulates the mechanics and associated geometric changes of a Sphincter of Oddi (SO) of the subject during contraction and relaxation.

16. The method of claim 13, wherein the step of inserting is performed endoscopically.

17. The method of claim 13, wherein the targeted duct is wholly or partially compressed or obstructed by a cancerous mass or tumor.

18. The method of claim 17, further comprising administering to the subject a treatment for the cancerous mass or tumor (e.g., chemotherapy or chemoradiotherapy).

19. The stent of claim 1, wherein an interior surface that defines the lumen of the first region comprises two or more ASMA unit cells.

20. The stent of claim 10, wherein each ASMA unit cell has a wavelength of 35 mm, 40 mm, 50 mm, or 60 mm.
